SEQUENCE LISTING (63 sequences)

SEQ ID NO: 1 - protein, 5 aa, synthetic construct
  REGION 1..5: note = Description of Sequence: Antibody component
NFGMH

SEQ ID NO: 2 - protein, 17 aa, synthetic construct
  REGION 1..17: note = Description of Sequence: Antibody component
YITSGGSSINYADTVKG

SEQ ID NO: 3 - protein, 13 aa, synthetic construct
  REGION 1..13: note = Description of Sequence: Antibody component
GGTGTRSLYYFDY

SEQ ID NO: 4 - protein, 11 aa, synthetic construct
  REGION 1..11: note = Description of Sequence: Antibody component
RASQDIGNFLN

SEQ ID NO: 5 - protein, 7 aa, synthetic construct
  REGION 1..7: note = Description of Sequence: Antibody component
YTSRLQS

SEQ ID NO: 6 - protein, 9 aa, synthetic construct
  REGION 1..9: note = Description of Sequence: Antibody component
QQGKTLPYT

SEQ ID NO: 7 - protein, 122 aa, synthetic construct
  REGION 1..122: note = Description of Sequence: Antibody component
QVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSS

SEQ ID NO: 8 - protein, 108 aa, synthetic construct
  REGION 1..108: note = Description of Sequence: Antibody component
DIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKD

SEQ ID NO: 9 - protein, 245 aa, synthetic construct
  REGION 1..245: note = Description of Sequence: Antibody component
QVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSSGGGGSGGGGSGGGGSDIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKD

SEQ ID NO: 10 - protein, 245 aa, synthetic construct
  REGION 1..245: note = Description of Sequence: Antibody component
DIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKDGGGGSGGGGSGGGGSQVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSS

SEQ ID NO: 11 - DNA, 735 bp, synthetic construct
  misc_feature 1..735: note = Description of Sequence: Coding sequence for antibody component
  source 1..735: mol_type = other DNA
caggtgcagctggtggagagcggcggcggcgtggtgcagcccggcagaagcctgagactgagctgcgccgccagcggcttcaccttcagcaacttcggcatgcactgggtgagacaggcccccggcaagggcctggagtgggtggcctacatcaccagcggcggcagcagcatcaactacgccgacaccgtgaagggcagattcaccatcagcagagacaacagcaagaacaccctgtacctgcagatgaacagcctgagagccgaggacaccgccgtgtactactgcaccagaggcggcaccggcaccagaagcctgtactacttcgactactggggccagggcaccaccgtgaccgtgagcagcggtggaggtggatcaggtggaggtggatctggtggaggtggatctgacatccagatgacccagagccccagaagcctgagcgccagcgtgggcgacagagtgaccatcacctgcagagccagccaggacatcggcaacttcctgaactggtaccaacagaagcccggcaaggcccccaagctgctgatctactacaccagtagactgcagagcggcgtgcccagcagatttagtggtagcggtagcggcaccgactacaccctgaccatcagcagcctgcagcccgaggacttcgccacctactactgccagcagggcaagaccctgccctacaccttcggcggcggcaccaaggtggagatcaaggac

SEQ ID NO: 12 - protein, 15 aa, synthetic construct
  REGION 1..15: note = Description of Sequence: Antibody component
YISSGGSSINYADTV

SEQ ID NO: 13 - protein, 122 aa, synthetic construct
  REGION 1..122: note = Description of Sequence: Antibody component
QVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGTTVTVSS

SEQ ID NO: 14 - protein, 107 aa, synthetic construct
  REGION 1..107: note = Description of Sequence: Antibody component
DIQMTQSPSSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIK

SEQ ID NO: 15 - protein, 122 aa, synthetic construct
  REGION 1..122: note = Description of Sequence: Antibody component
DVQLVESGGGLVQPGGSRKLSCAASGFTFSNFGMHWVRQAPEKGLEWVAYISSGGSSINYADTVKGRFTISRDNPKNTLFLQMTSLRSEDTAIYYCTRGGTGTRSLYYFDYWGQGATLIVSS

SEQ ID NO: 16 - protein, 107 aa, synthetic construct
  REGION 1..107: note = Description of Sequence: Antibody component
DIQMTQSPSSLSASVGDRVTITCRASQDIGNFLNWYQQKPGKAPKLLLYYTSRLQSGVPSRFSGSGSGTDYTLTISSLQPEDFATYYCQQGKTLPYTFGGGTKVEIK

SEQ ID NO: 17 - protein, 19 aa, synthetic construct
  REGION 1..19: note = Description of Sequence: Antibody component
GGGGSGGGGSGGGSGGGGS

SEQ ID NO: 18 - protein, 15 aa, synthetic construct
  REGION 1..15: note = Description of Sequence: Antibody component
GGGGSGGGGSGGGGS

SEQ ID NO: 19 - protein, 24 aa, synthetic construct
  REGION 1..24: note = Description of Sequence: Antibody component
GGGGSGGGGSGGGGSGGGSGGGGS

SEQ ID NO: 20 - protein, 29 aa, synthetic construct
  REGION 1..29: note = Description of Sequence: Antibody component
GGGGSGGGGSGGGGSGGGGSGGGSGGGGS

SEQ ID NO: 21 - protein, 5 aa, synthetic construct
  REGION 1..5: note = Description of Sequence: Antibody component
GGGGS

SEQ ID NO: 22 - protein, 10 aa, synthetic construct
  REGION 1..10: note = Description of Sequence: Antibody component
GGGGSGGGGS

SEQ ID NO: 23 - protein, 235 aa, synthetic construct
  REGION 1..235: note = Description of Sequence: Antibody component
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 24 - protein, 247 aa, synthetic construct
  REGION 1..247: note = Description of Sequence: Antibody component
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 25 - protein, 220 aa, synthetic construct
  REGION 1..220: note = Description of Sequence: Antibody component
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 26 - DNA, 81 bp, synthetic construct
  misc_feature 1..81: note = Description of Sequence: Coding sequence for antibody component
  source 1..81: mol_type = other DNA
ttttgggtgctggtggtggttggtggagtcctggcttgctatagcttgctagtaacagtggcctttattattttctgggtg

SEQ ID NO: 27 - protein, 218 aa, synthetic construct
  REGION 1..218: note = Description of Sequence: Antibody component
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 28 - DNA, 117 bp, synthetic construct
  misc_feature 1..117: note = Description of Sequence: Coding sequence for antibody component
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 29 - protein, 164 aa, synthetic construct
  REGION 1..164: note = Description of Sequence: Antibody component
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 112 aa, synthetic construct
  REGION 1..112: note = Description of Sequence: Antibody component
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 339 bp, synthetic construct
  misc_feature 1..339: note = Description of Sequence: Coding sequence for antibody component
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 32 - protein, 22 aa, synthetic construct
  REGION 1..22: note = Description of Sequence: Antibody component
QNQLYNELNLGRREEYDVLDKR

SEQ ID NO: 33 - DNA, 66 bp, synthetic construct
  misc_feature 1..66: note = Description of Sequence: Coding sequence for antibody component
  source 1..66: mol_type = other DNA
cagaaccagctctataacgagctcaatctaggacgaagagaggagtacgatgttttggacaagaga

SEQ ID NO: 34 - protein, 22 aa, synthetic construct
  REGION 1..22: note = Description of Sequence: Antibody component
QNQLFNELNLGRREEFDVLDKR

SEQ ID NO: 35 - DNA, 66 bp, synthetic construct
  misc_feature 1..66: note = Description of Sequence: Coding sequence for antibody component
  source 1..66: mol_type = other DNA
cagaaccagctctttaacgagctcaatctaggacgaagagaggagttcgatgttttggacaagaga

SEQ ID NO: 36 - protein, 23 aa, synthetic construct
  REGION 1..23: note = Description of Sequence: Antibody component
QEGLYNELQKDKMAEAYSEIGMK

SEQ ID NO: 37 - DNA, 69 bp, synthetic construct
  misc_feature 1..69: note = Description of Sequence: Coding sequence for antibody component
  source 1..69: mol_type = other DNA
caggaaggcctgtacaatgaactgcagaaagataagatggcggaggcctacagtgagattgggatgaaa

SEQ ID NO: 38 - protein, 23 aa, synthetic construct
  REGION 1..23: note = Description of Sequence: Antibody component
QEGLFNELQKDKMAEAFSEIGMK

SEQ ID NO: 39 - DNA, 69 bp, synthetic construct
  misc_feature 1..69: note = Description of Sequence: Coding sequence for antibody component
  source 1..69: mol_type = other DNA
caggaaggcctgttcaatgaactgcagaaagataagatggcggaggccttcagtgagattgggatgaaa

SEQ ID NO: 40 - protein, 22 aa, synthetic construct
  REGION 1..22: note = Description of Sequence: Antibody component
HDGLYQGLSTATKDTYDALHMQ

SEQ ID NO: 41 - DNA, 66 bp, synthetic construct
  misc_feature 1..66: note = Description of Sequence: Coding sequence for antibody component
  source 1..66: mol_type = other DNA
cacgatggcctttaccagggtctcagtacagccaccaaggacacctacgacgcccttcacatgcag

SEQ ID NO: 42 - protein, 22 aa, synthetic construct
  REGION 1..22: note = Description of Sequence: Antibody component
HDGLFQGLSTATKDTFDALHMQ

SEQ ID NO: 43 - DNA, 66 bp, synthetic construct
  misc_feature 1..66: note = Description of Sequence: Coding sequence for antibody component
  source 1..66: mol_type = other DNA
cacgatggccttttccaggggctcagtacagccaccaaggacaccttcgacgcccttcacatgcag

SEQ ID NO: 44 - protein, 112 aa, synthetic construct
  REGION 1..112: note = Description of Sequence: Antibody component
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 45 - DNA, 336 bp, synthetic construct
  misc_feature 1..336: note = Description of Sequence: Coding sequence for antibody component
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 46 - DNA, 123 bp, synthetic construct
  misc_feature 1..123: note = Description of Sequence: Coding sequence for antibody component
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 47 - protein, 255 aa, synthetic construct
  REGION 1..255: note = Description of Sequence: Antibody component
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 48 - DNA, 126 bp, synthetic construct
  misc_feature 1..126: note = Description of Sequence: Coding sequence for antibody component
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 49 - protein, 487 aa, synthetic construct
  REGION 1..487: note = Description of Sequence: Antibody component
MALPVTALLLPLALLLHAEQVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSSGGGGSGGGGSGGGGSDIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKAAAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - DNA, 1467 bp, synthetic construct
  misc_feature 1..1467: note = Description of Sequence: Coding sequence for antibody component
  source 1..1467: mol_type = other DNA
atggctctcccagtgactgccctactgcttcccctagcgcttctcctgcatgctgaacaggtgcagctggtggagagcggcggcggcgtggtgcagcccggcagaagcctgagactgagctgcgccgccagcggcttcaccttcagcaacttcggcatgcactgggtgagacaggcccccggcaagggcctggagtgggtggcctacatcaccagcggcggcagcagcatcaactacgccgacaccgtgaagggcagattcaccatcagcagagacaacagcaagaacaccctgtacctgcagatgaacagcctgagagccgaggacaccgccgtgtactactgcaccagaggcggcaccggcaccagaagcctgtactacttcgactactggggccagggcaccaccgtgaccgtgagcagcggtggaggtggatcaggtggaggtggatctggtggaggtggatctgacatccagatgacccagagccccagaagcctgagcgccagcgtgggcgacagagtgaccatcacctgcagagccagccaggacatcggcaacttcctgaactggtaccaacagaagcccggcaaggcccccaagctgctgatctactacaccagtagactgcagagcggcgtgcccagcagatttagtggtagcggtagcggcaccgactacaccctgaccatcagcagcctgcagcccgaggacttcgccacctactactgccagcagggcaagaccctgccctacaccttcggcggcggcaccaaggtggagatcaaggacgcggccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - protein, 8 aa, synthetic construct
  REGION 1..8: note = Description of Sequence: Antibody component
DYKDDDDK

SEQ ID NO: 52 - protein, 496 aa, synthetic construct
  REGION 1..496: note = Description of Sequence: Antibody component
MALPVTALLLPLALLLHAEQVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSSGGGGSGGGGSGGGGSDIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKDDYKDDDDKAAAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 1491 bp, synthetic construct
  misc_feature 1..1491: note = Description of Sequence: Coding sequence for antibody component
  source 1..1491: mol_type = other DNA
atggctctcccagtgactgccctactgcttcccctagcgcttctcctgcatgctgaacaggtgcagctggtggagagcggcggcggcgtggtgcagcccggcagaagcctgagactgagctgcgccgccagcggcttcaccttcagcaacttcggcatgcactgggtgagacaggcccccggcaagggcctggagtgggtggcctacatcaccagcggcggcagcagcatcaactacgccgacaccgtgaagggcagattcaccatcagcagagacaacagcaagaacaccctgtacctgcagatgaacagcctgagagccgaggacaccgccgtgtactactgcaccagaggcggcaccggcaccagaagcctgtactacttcgactactggggccagggcaccaccgtgaccgtgagcagcggtggaggtggatcaggtggaggtggatctggtggaggtggatctgacatccagatgacccagagccccagaagcctgagcgccagcgtgggcgacagagtgaccatcacctgcagagccagccaggacatcggcaacttcctgaactggtaccaacagaagcccggcaaggcccccaagctgctgatctactacaccagtagactgcagagcggcgtgcccagcagatttagtggtagcggtagcggcaccgactacaccctgaccatcagcagcctgcagcccgaggacttcgccacctactactgccagcagggcaagaccctgccctacaccttcggcggcggcaccaaggtggagatcaaggacgactacaaagacgatgacgacaaggcggccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 54 - protein, 157 aa, synthetic construct
  REGION 1..157: note = Description of Sequence: Antibody component
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 55 - DNA, 471 bp, synthetic construct
  misc_feature 1..471: note = Description of Sequence: Coding sequence for antibody component
  source 1..471: mol_type = other DNA
tacttcggcaagctggagagcaagctgagcgtgatcaggaacctgaacgaccaggtgctgttcatcgaccagggcaacaggcccctgttcgaggacatgaccgacagcgactgcagggacaacgcccccaggaccatcttcatcatcagcatgtacaaggacagccagcccaggggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagaggagcgtgcccggccacgacaacaagatgcagttcgagagcagcagctacgagggctacttcctggcctgcgagaaggagagggacctgttcaagctgatcctgaagaaggaggacgagctgggcgacaggagcatcatgttcaccgtgcagaacgaggac

SEQ ID NO: 56 - protein, 153 aa, Homo sapiens
KIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 57 - protein, 160 aa, Homo sapiens
REAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 58 - protein, 152 aa, Homo sapiens
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 59 - protein, 152 aa, Mus musculus
GRETPDFGEVFDLDQQVWIFRNQALVTVPRSHRVTPVSVTILPCKYPESLEQDKGIAIYLGIQNPDKCLFCKEVNGHPTLLLKEEKILDLYHHPEPMKPFLFYHTRTGGTSTFESVAFPGHYIASSKTGNPIFLTSKKGEYYNINFNLDIKS

SEQ ID NO: 60 - protein, 153 aa, Mus musculus
SSQSPRNYRVHDSQQMVWVLTGNTLTAVPASNNVKPVILSLIACRDTEFQDVKKGNLVFLGIKNRNLCFCCVEMEGKPTLQLKEVDIMNLYKERKAQKAFLFYHGIEGSTSVFQSVLYPGWFIATSSIERQTIILTHQRGKLVNTNFYIESEK

SEQ ID NO: 61 - protein, 152 aa, Mus musculus; identical to SEQ ID NO: 59

SEQ ID NO: 62 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 63 - protein, 158 aa, Mus musculus
SIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

(19) United States
(12) Patent Application Publication
(10) Pub. No.: US 2024/0042031 A1
(43) Pub. Date: Feb. 8, 2024

(54) ANTIGEN RECOGNIZING RECEPTORS TARGETING GD3 GANGLIOSIDE AND USES THEREOF

(71) Applicant: MEMORIAL SLOAN-KETTERING CANCER CENTER, New York, NY (US)

(72) Inventors: Christopher S. Hackett, New York, NY (US); Renier J. Brentjens, Short Hills, NJ (US); Jedd D. Wolchok, New York, NY (US); Paul B. Chapman, New York, NY (US); Sarwish Rafiq, Dunwoody, GA (US); Terence Purdon, New York, NY (US)

(73) Assignee: MEMORIAL SLOAN-KETTERING CANCER CENTER, New York, NY (US)

(21) Appl. No.: 18/476,870

(22) Filed: Sep. 28, 2023

Related U.S. Application Data
(63) Continuation of application No. PCT/US22/22427, filed on Mar. 30, 2022.
(60) Provisional application No. 63/214,212, filed on Jun. 23, 2021.
(60) Provisional application No. 63/168,543, filed on Mar. 31, 2021.

(51) Int. Cl. A61K 39/00; C07K 16/30; C07K 14/725; C07K 14/705; C07K 14/54; A61P 35/00; C12N 5/0783

(52) U.S. Cl. CPC A61K 39/4632; C07K 16/3084; C07K 14/7051; C07K 14/70521; C07K 14/70578; C07K 14/70575; C07K 14/54; A61P 35/00; A61K 39/4611; C12N 5/0636; C07K 2317/622; C07K 2317/24; C07K 2319/02; C07K 2319/03

(57) ABSTRACT

The presently disclosed subject matter provides for antigen-recognizing receptors that specifically target GD3 and cells comprising such GD3-targeted antigen-recognizing receptors. The presently disclosed subject matter further provides uses of the GD3-targeted antigen-recognizing receptors for treatment.

### CROSS-REFERENCE TO RELATED APPLICATIONS

This application is a continuation application of International Patent Application No. PCT/US2022/022427, filed Mar. 30, 2022, which claims priority to each of U.S. Provisional Application No. 63/168,543, filed Mar. 31, 2021, and U.S. Provisional Application No. 63/214,212, filed Jun. 23, 2021, the content of each of which is incorporated by reference in its entirety, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Sep. 28, 2023, is entitled “072734.1475_ST26.xml”, and is 88,863 bytes in size.

### 1. INTRODUCTION

The presently disclosed subject matter provides methods and compositions for immunotherapies. It relates to antigen-recognizing receptors (e.g., chimeric antigen receptors (CARs)) that specifically target GD3 ganglioside, cells comprising such receptors, and methods of using such cells for treatments.

### 2. BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Treatment for immunotherapy-resistant melanoma, metastatic osteosarcomas, and small cell lung cancer has seen little improvement over the past several decades, highlighting a need for novel therapies. Chimeric antigen receptor (CAR) T cells are a powerful emerging approach to cancer therapy in which a patient's T cells are mobilized to kill tumor cells through ex-vivo genetic introduction of a novel receptor. CAR T cells targeting CD19 showed 80-90% response rates in patients with relapsed or refractory acute lymphoblastic leukemia (Maude et al. N Engl J Med (2014); 371:1507-1517; Brentjens et al., Sci Transl Med (2013); 5, 177ra138), showed additional efficacy in lymphoma (Schuster et al. N Engl J Med (2017); 377, 2545-2554). Though CAR T cells have demonstrated clinical benefit in hematological malignancies, they have generally not demonstrated clinical efficacy in solid tumors to date.

One cause of CAR T cell failure in solid tumors is a suppressive solid tumor microenvironment (Lim et al., Cell (2017); 168, 724-740). Accordingly, there are needs for novel therapeutic strategies to design CARs for solid tumors including sarcoma, melanoma, and lung cancer, and for strategies capable of inducing potent cancer eradication with minimal toxicity and immunogenicity.

### 3. SUMMARY OF THE INVENTION

The presently disclosed subject matter provides antigen-recognizing receptors that specifically target ganglioside GD3 and cells comprising such GD3-targeted antigen-recognizing receptors. The presently disclosed subject matter further provides uses of the GD3-targeted antigen-recognizing receptors for treatment. The presently disclosed subject matter provides an antigen-recognizing receptor comprising an extracellular antigen-binding domain, a transmembrane domain, and an intracellular signaling domain, wherein the extracellular antigen-binding domain specifically binds to GD3 ganglioside.

In certain embodiments, the extracellular antigen-binding domain is a single-chain variable fragment (scFv), a Fab, or a F(ab)₂. In certain embodiments, one or more of the scFv, Fab and F(ab)₂ are comprised in a fusion protein with a heterologous sequence to form the extracellular antigen-binding domain. In certain embodiments, the extracellular antigen-binding domain is a humanized scFv.

In certain embodiments, the extracellular antigen-binding domain comprises a heavy chain variable region and a light chain variable region, wherein
a) the heavy chain variable region comprises a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and/or
b) the light chain variable region comprises a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof.

In certain embodiments, the heavy chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3.

In certain embodiments, the light chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the heavy chain variable region comprises a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and the light chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7. In certain embodiments, the heavy chain variable region comprises the amino acid sequence set forth in SEQ ID NO: 7.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or amino acids 1 to 107 of SEQ ID NO: 8. In certain embodiments, the light chain variable region comprises the amino acid sequence set forth in SEQ ID NO: 8 or amino acids 1 to 107 of SEQ ID NO: 8.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7; and the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or amino acids 1 to 107 of SEQ ID NO: 8.

In certain embodiments, the heavy chain variable region comprising the amino acid sequence set forth in SEQ ID NO: 7; and the light chain variable region comprises the amino acid sequence set forth in SEQ ID NO: 8. In certain embodiments, the heavy chain variable region comprising the amino acid sequence set forth in SEQ ID NO: 7; and the light chain variable region comprises amino acids 1 to 107 of SEQ ID NO: 8.

In certain embodiments, the extracellular antigen-binding domain comprises a linker between the heavy chain variable region and the light chain variable region. In certain embodiments, the linker consists of the amino acid sequence set forth in SEQ ID NO: 17, SEQ ID NO: 18, SEQ ID NO: 19, SEQ ID NO: 20, SEQ ID NO: 21, or SEQ ID NO: 22. In certain embodiments, the linker consists of the amino acid sequence set forth in SEQ ID NO: 18.

In certain embodiments, a signal peptide is covalently joined to the 5′ terminus of the extracellular antigen-binding domain.

In certain embodiments, the heavy chain variable region and the light chain variable region are positioned from the N- to the C-terminus: V_{H}-V_{L}.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the transmembrane domain comprises a CD28 polypeptide.

In certain embodiments, the intracellular signaling domain comprises a CD3ζ polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide or a 4-1BB polypeptide. In certain embodiments, the at least one co-stimulatory signaling region comprises a CD28 polypeptide.

In certain embodiments, the antigen-recognizing receptor is a chimeric antigen receptor (CAR) or a T-cell Receptor (TCR) like fusion protein. In certain embodiments, the antigen-recognizing receptor is a CAR.

In certain embodiments, the antigen-recognizing receptor is recombinantly expressed. In certain embodiments, the antigen-recognizing receptor is expressed from a vector. In certain embodiments, the vector is a retroviral vector. In certain embodiments, the retroviral vector is a γ-retroviral vector or a lentiviral vector.

The presently disclosed subject matter provides cells comprising a presently disclosed antigen-recognizing receptor. In certain embodiments, the cell is transduced with the antigen-recognizing receptor. In certain embodiments, the antigen-recognizing receptor is constitutively expressed on the surface of the cell.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which a lymphoid cell may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell. In certain embodiments, the T cell is a CD4⁺ T cell. In certain embodiments, the T cell is a CD8⁺ T cell.

In certain embodiments, the cell is engineered to express at least one cytokine or a fragment thereof. In certain embodiments, the cytokine is selected from the group consisting of IL-18, IL-36, IL-33, IL-12, IL-21, and IL-2. In certain embodiments, the cell comprises an exogenous IL-18 polypeptide, an exogenous IL-36 polypeptide, an IL-33 polypeptide, or a combination thereof. In certain embodiments, the cell comprises an exogenous IL-36γ polypeptide.

In certain embodiments, the cell comprises a soluble antibody, a soluble antigen-binding fragment, or a fusion protein, which binds to a polypeptide having immunosuppressive activity or immunostimulatory activity. In certain embodiments, the antibody, antigen-binding fragment, or fusion protein is selected from the group consisting of monoclonal antibodies, single-chain variable fragments (scFvs), scFv-Fc fusion proteins, bispecific antibodies, minibodies, and BiTE. In certain embodiments, the antigen-binding fragment is a single chain variable fragment (scFv). In certain embodiments, the fusion protein is a scFv-Fc-fusion protein. In certain embodiments, the polypeptide having immunosuppressive activity is selected from the group consisting of CD47, PD-1, CTLA-4, BTLA, LAG-3, 2B4, CD47, SIRPα, PD-L1, PD-L2, TNFRSF14, CD48, and FGL-1. In certain embodiments, the soluble antibody, antigen-binding fragment thereof, or fusion protein is an antagonist antibody, antigen-binding fragment thereof, or fusion protein. In certain embodiments, the soluble antibody, antigen-binding fragment thereof, or fusion protein is an antagonist antibody, antigen-binding fragment thereof, or fusion protein that binds to PD-1 or CTLA-4. In certain embodiments, the soluble antibody, antigen-binding fragment thereof, or fusion protein is an antagonist scFv that binds to PD-1 or an antagonist scFv-Fc fusion protein that binds to CTLA-4.

In certain embodiments, the polypeptide having immunostimulatory activity is selected from the group consisting of CD28, CD40, OX-40, 4-1BB, B7-1, B7-2, CD40L, OX-40L, 4-1BBL, GITR, and GITRL. In certain embodiments, the soluble antibody, antigen-binding fragment thereof, or fusion protein is an agonist antibody, antigen-binding fragment thereof, or fusion protein. In certain embodiments, the soluble antibody, antigen-binding fragment thereof, or fusion protein is an agonist antibody, antigen-binding fragment thereof, or fusion protein that binds to CD40. In certain embodiments, the soluble antibody, antigen-binding fragment thereof, or fusion protein is an agonist scFv-Fc fusion protein that binds to CD40.

In certain embodiments, the cell comprises an exogenous IL-18 polypeptide and a soluble antibody, a soluble antigen-binding fragment thereof, or a fusion protein is agonist, which binds to PD-1. In certain embodiments, the cell comprises an exogenous IL-36 polypeptide and a soluble agonist scFv that binds to PD-1. In certain embodiments, the cell comprises an exogenous IL-18 polypeptide, an exogenous IL-36 polypeptide, and a soluble agonist scFv that binds to PD-1. In certain embodiments, the cell comprises an exogenous IL-36γ polypeptide. In certain embodiments, the cell comprises an exogenous IL-18 polypeptide and a soluble agonist scFv-Fc fusion protein that binds to CD40.

In certain embodiments, the cell further comprises an exogenous CD40L. In certain embodiments, the cell comprises an exogenous IL-18 polypeptide and an exogenous CD40L. In certain embodiments, the cell comprises an exogenous IL-36 polypeptide and an exogenous CD40L.

The presently disclosed subject matter further provides nucleic acid molecules encoding presently disclosed antigen-recognizing receptors. The presently disclosed subject matter further provides vectors comprising the presently disclosed nucleic acid molecules. In certain embodiments, the vector is a viral vector. In certain embodiments, the vector is a retroviral vector. In certain embodiments, the retroviral vector is a γ-retroviral vector or a lentiviral vector.

In addition, the presently disclosed subject matter provides host cells expressing the nucleic acid molecule disclosed herein. In certain embodiments, the host cell is a T cell.

The presently disclosed subject matter further provides compositions comprising the cells disclosed herein. In certain embodiments, the composition is a pharmaceutical composition further comprising a pharmaceutically acceptable carrier.

The presently disclosed subject matter further provides methods of reducing tumor burden in a subject. In certain embodiments, the method comprises administering an effective amount of presently disclosed cells or composition to the subject. In certain embodiments, the method reduces the number of tumor cells, reduces tumor size, and/or eradicates the tumor in the subject. The presently disclosed subject matter further provides methods of increasing or lengthening survival of a subject having a tumor or neoplasm. In certain embodiments, the method comprises administering an effective amount of presently disclosed cells or composition to the subject. The presently disclosed subject matter further provides methods of treating and/or preventing a tumor or neoplasm in a subject.

In certain embodiments, the tumor is associated with ganglioside GD3. In certain embodiments, the tumor is a cancer. In certain embodiments, the tumor is selected from the group consisting of sarcoma, Merkel cell carcinoma (MCC), lung cancer, and melanoma. In certain embodiments, the lung cancer is small cell lung cancer (SCLC) or non-small cell lung cancer. In certain embodiments, the non-small cell lung cancer comprises lung adenosarcoma.

In certain embodiments, the tumor is sarcoma. In certain embodiments, the sarcoma is selected from the group consisting of bone sarcoma, soft tissue sarcoma, fibrosarcoma, myxosarcoma, chondrosarcoma, chordoma, angiosarcoma, endotheliosarcoma, lymphangiosarcoma, lymphangioendotheliosarcoma, synovioma, mesothelioma, Ewing's tumor (or Ewing's sarcoma), leiomyosarcoma, rhabdomyosarcoma, and undifferentiated pleiomorphic sarcoma.

In certain embodiments, the tumor is selected from the group consisting of melanoma, Merkel cell carcinoma (MCC), bone sarcoma, soft tissue sarcoma, and small cell lung cancer (SCLC). In certain embodiments, the bone sarcoma comprises osteosarcoma. In certain embodiments, the soft tissue sarcoma is selected from the group consisting of liposarcoma, myxofibrosarcoma, undifferentiated pleiomorphic sarcoma, and leiomyosarcoma.

The presently disclosed subject matter further provides methods for producing a presently disclosed cell comprising a GD3-targeted antigen-recognizing receptor. In certain embodiments, the method comprises introducing into the cell a nucleic acid molecule that encodes the antigen-recognizing receptor.

In addition, the presently disclosed subject matter provides kits for reducing tumor burden in a subject, treating and/or preventing a tumor or neoplasm in a subject, and/or increasing or lengthening survival of a subject having a tumor or neoplasm. In certain embodiments, the kit comprises the cell described herein. In certain embodiments, the kit further comprises written instructions for using the cell for reducing tumor burden in a subject, treating and/or preventing a tumor or neoplasm in a subject, and/or increasing or lengthening survival of a subject having a tumor or neoplasm.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A-1F illustrate GD3-targeted CAR T cells killing human melanoma, small cell lung cancer (SCLC), and osteosarcoma cells. FIG. 1A depicts GD3 expression on human tumor cell lines. The expression was measured by flow cytometry. Grey, left-most curves represent isotype control. FIG. 1B depicts proliferation of hR24v CAR T cells when co-cultured with GD3+ small cell lung cancer cells. 4H11 CAR binds to MUC16, and was used as a negative control. FIG. 1C depicts cytokine release from hR24v CAR T cells when co-cultured with GD3+ melanoma cells. 1928z binds to CD19, and was used as a negative control. FIG. 1D depicts in vitro cytotoxicity of hR24v CAR T cells (solid lines with round points) as compared to off-target control (4H11) CAR T cells (dashed lines with square points). The in vitro cytotoxicity was measured by bioluminescence cell killing assays. FIG. 1E depicts experimental design and in vivo antitumor efficacy of hR24v CAR T cells against melanoma as compared to off-target 4H11 CAR T cells. The efficacy was measured at 31 days post-CAR T cell administration. FIG. 1F depicts in vitro cytotoxicity of hR24v CAR T cells (full circles) as compared to off-target controls (4H11 or CD19) CAR T cells (full squares).

FIG. 2 illustrates exemplary constructs of hR24v CAR disclosed herein. “CD28 tm/signaling” represents a transmembrane domain of CD28 fused to a signaling (intracellular) domain of CD28.

FIG. 3 illustrates secretion profiles of IL-18 by T cells. “hR24v” represents T cells comprising a presently disclosed GD3-targeted CAR. “hR24v-IL18” represents T cells comprising a presently disclosed GD3-targeted CAR and a secretable IL-18 polypeptide. “hR24v-E27-IL18” represents T cells comprising a presently disclosed GD3-targeted CAR, a secretable IL-18 polypeptide, and an anti-PD1 scFv E27. “hR24v1xx-E27-IL18” represents T cells comprising a presently disclosed GD3-targeted CAR comprising a modified CD3ζ polypeptide “1xx”, a secretable IL-18 polypeptide, and an anti-PD1 scFv E27.

### 5. DETAILED DESCRIPTION OF THE INVENTION

The presently disclosed subject matter provides antigen-recognizing receptors (e.g., a chimeric antigen receptors (CAR) or a T-cell receptor (TCR) like fusion molecule) that specifically target GD3. The presently disclosed subject matter further provides cells comprising such receptors. The cells can be immunoresponsive cells, e.g., genetically modified immunoresponsive cells (e.g., T cells or NK cells). The presently disclosed subject matter also provides methods of using such cells for treatments, e.g., for treating and/or preventing a tumor or neoplasm (e.g., sarcoma (e.g., bone sarcoma (e.g., osteosarcoma), soft tissue sarcoma (e.g., liposarcoma, myxofibrosarcoma, and leiomyosarcoma)), melanoma, and small cell lung cancer (SCLC)).

Non-limiting embodiments of the present disclosure are described by the present specification and Examples.

For purposes of clarity of disclosure and not by way of limitation, the detailed description is divided into the following subsections:
5.1. Definitions;
5.2. GD3;
5.3. Antigen-Recognizing Receptors;
5.4. Cells;
5.5. Compositions and Vectors;
5.6. Polypeptides;
5.7. Formulations and Administration;
5.8. Methods of Treatment; and
5.9. Kits

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when a CAR or a TCR like fusion molecule binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., GD3). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The CDRs can also be numbered according to the IMGT numbering system, e.g., the IMGT numbering system accessible at http://www.imgt.org/IMGT_vquest/input. Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated according to the Kabat numbering system.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises the amino acid sequence set forth in SEQ ID NO: 17, which is provided below:

[SEQ ID NO: 17]
GGGGSGGGGSGGGSGGGGS

In certain embodiments, the linker comprise the amino acid sequence set forth in SEQ ID NO: 18, which is provided below:

[SEQ ID NO: 18]
GGGGSGGGGSGGGGS

In certain embodiments, the linker comprises the amino acid sequence set forth in SEQ ID NO: 19, which is provided below:

[SEQ ID NO: 19]
GGGGSGGGGSGGGGSGGGSGGGGS

In certain embodiments, the linker comprises the amino acid sequence set forth in SEQ ID NO: 20, which is provided below:

[SEQ ID NO: 20]
GGGGSGGGGSGGGGSGGGGSGGGSGGGGS

In certain embodiments, the linker comprises the amino acid sequence set forth in SEQ ID NO: 21, which is provided below:

[SEQ ID NO: 21]
GGGGS

In certain embodiments, the linker comprises the amino acid sequence set forth in SEQ ID NO: 22, which is provided below:

[SEQ ID NO: 22]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife eta., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chern (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

In certain embodiments, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, the term “immunosuppressive activity” is meant induction of signal transduction or changes in protein expression in a cell (e.g., an activated immunoresponsive cell) resulting in a decrease in an immune response. Non-limiting examples of polypeptides suppressing or decreasing an immune response via their binding include CD47, PD-1, CTLA-4, BTLA, LAG-3, 2B4, and their corresponding ligands (including, but not limited to, SIRPα, PD-L1, PD-L2, TNFRSF14, CD48, and FGL-1). Such polypeptides are present in the tumor microenvironment and inhibit immune responses to neoplastic cells. In certain embodiments, inhibiting, blocking, or antagonizing the interaction of immunosuppressive polypeptides and/or their ligands enhances the immune response of the immunoresponsive cell.

As used herein, the term “immunostimulatory activity” is meant induction of signal transduction or changes in protein expression in a cell (e.g., an activated immunoresponsive cell) resulting in an increase in an immune response. Immunostimulatory activity may include pro-inflammatory activity. Non-limiting examples of polypeptides stimulating or increasing an immune response via their binding include CD28, CD40, OX-40, 4-1BB, GITR, and their corresponding ligands, including B7-1, B7-2, CD40L, OX-40L, 4-1BBL, GITRL. Such polypeptides are present in the tumor microenvironment and activate immune responses to neoplastic cells. In certain embodiments, promoting, stimulating, or agonizing pro-inflammatory polypeptides and/or their ligands enhances the immune response of the immunoresponsive cell.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). The neoplasia can a primary tumor or primary cancer. In addition, the neoplasm can be in metastatic status.

By “receptor” is meant a polypeptide, or portion thereof, present on a cell membrane that selectively binds one or more ligand.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

By “specifically binds” or “specifically binds to” or “specifically target” is meant a polypeptide or a fragment thereof that recognizes and/or binds to a biological molecule of interest (e.g., a polypeptide, e.g., a GD3 ganglioside), but which does not substantially recognize and/or bind other molecules in a sample, for example, a biological sample, which naturally includes a presently disclosed ganglioside (e.g., a GD3 ganglioside).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 5.2. Ganglioside GD3

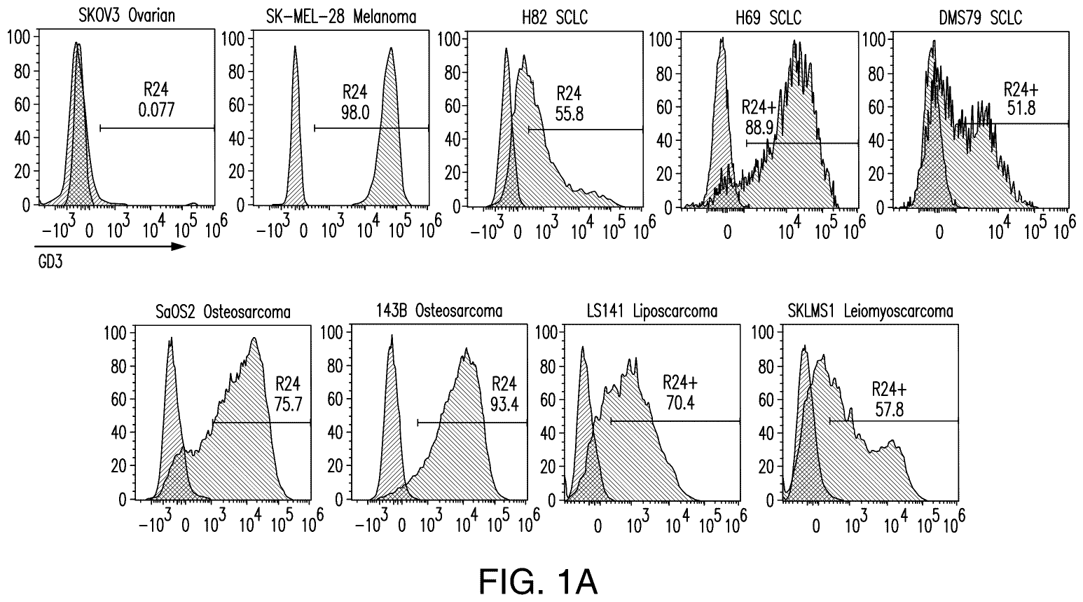

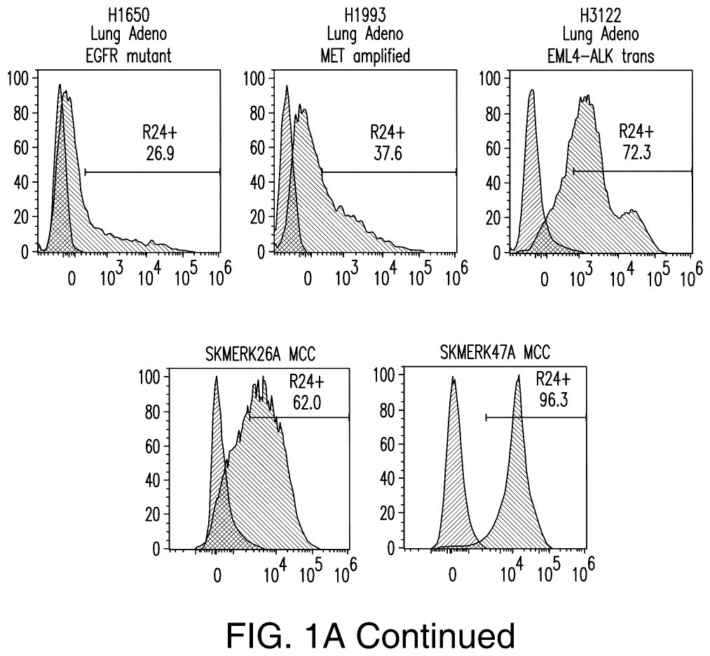

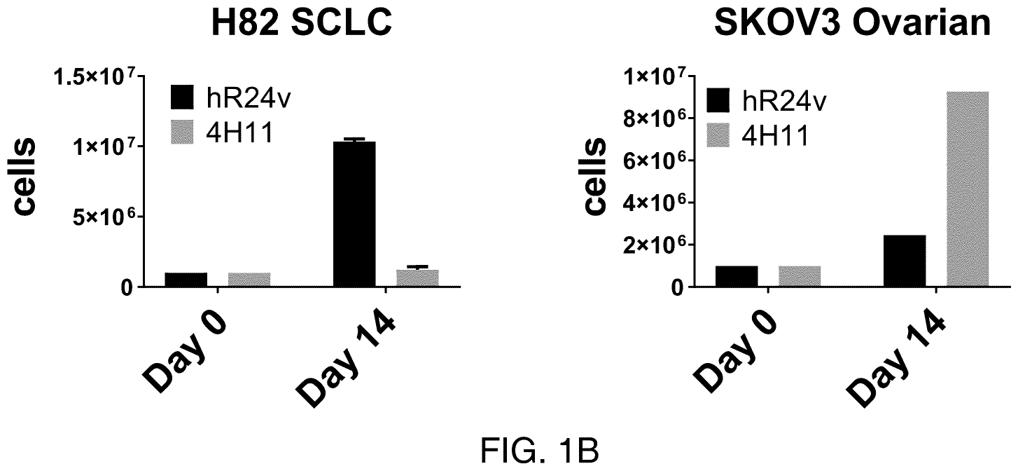

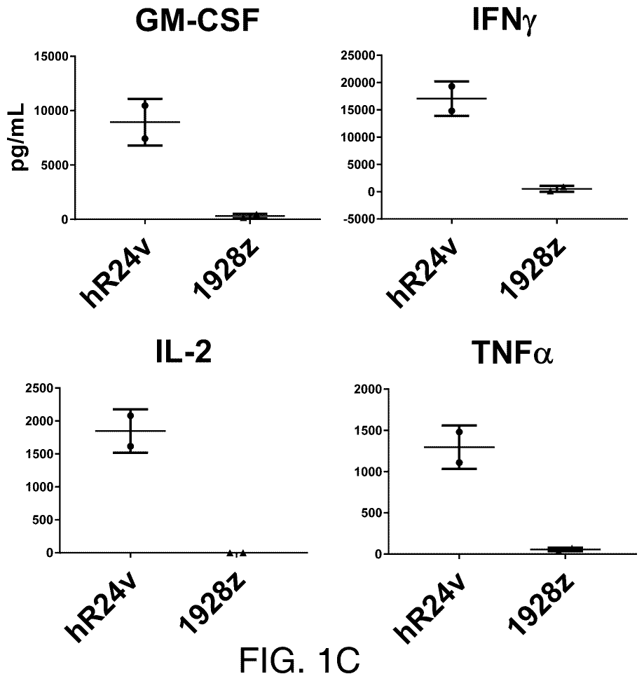

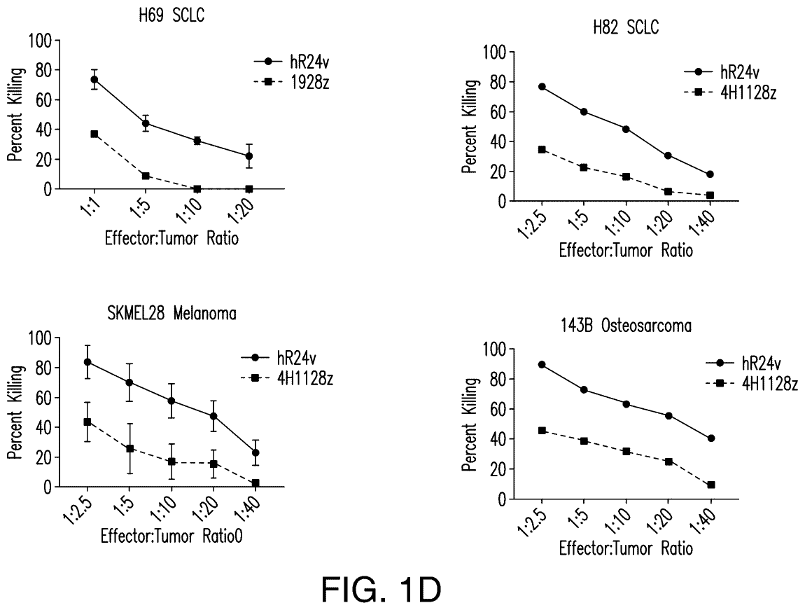

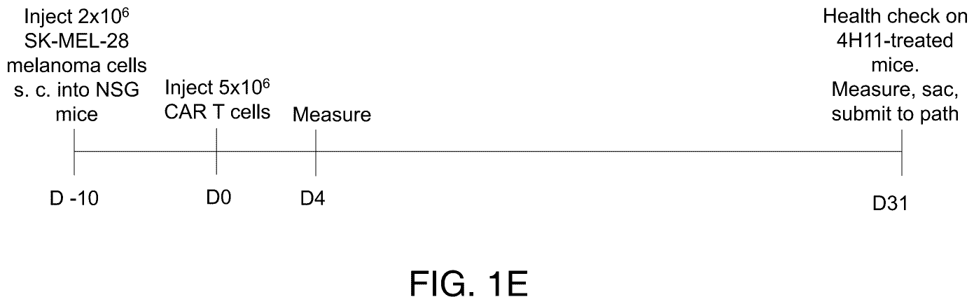

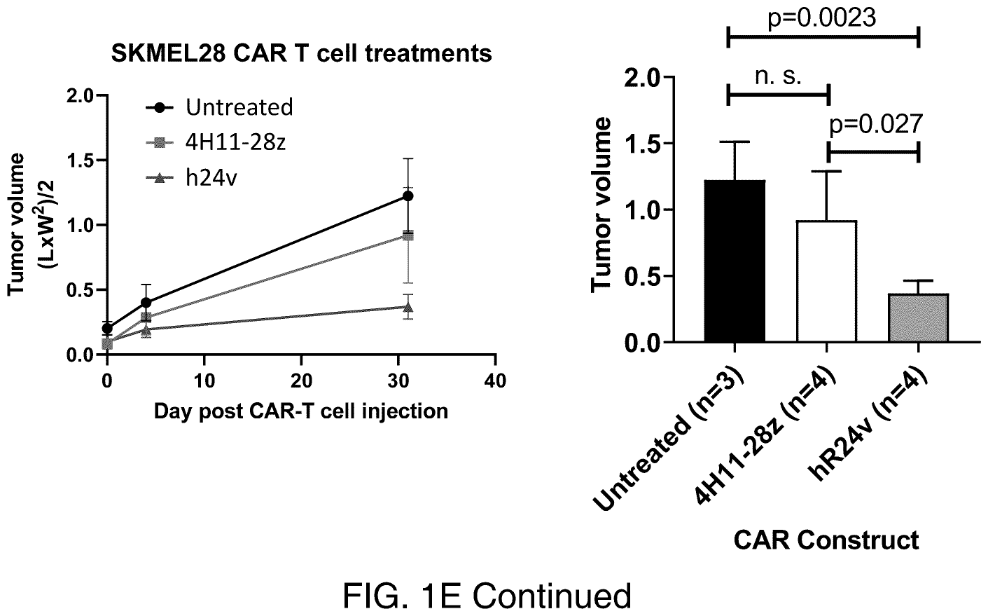

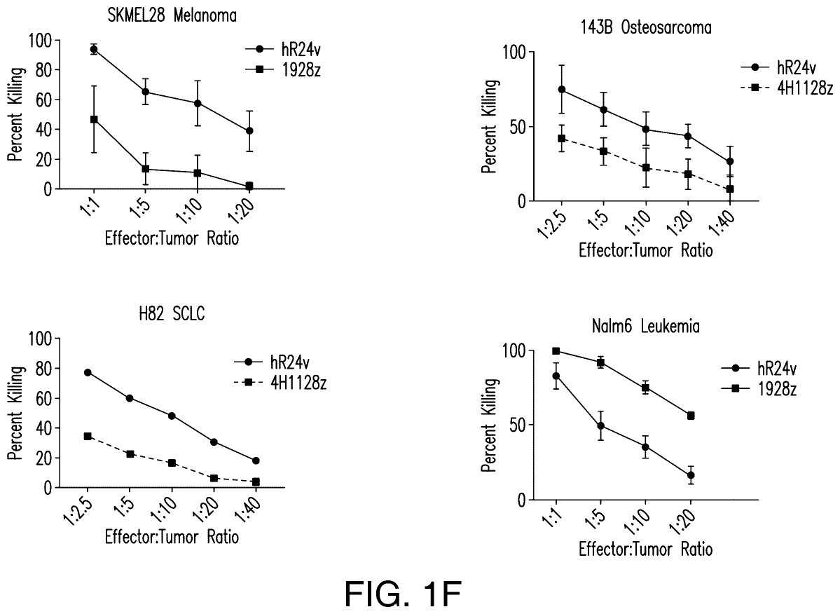

The ganglioside GD3 is expressed on over 80% of osteosarcomas (Dobrenkov et al., Pediatr Blood Cancer (2016) 63:1780-1785), providing a potential target for cellular therapies. GD3 is also expressed on 88% of soft tissue sarcomas (Chang et al., Cancer (1992); 70:633-638) and over 80% of melanomas, 60-80% of small cell lung cancer, and other neuroendocrine and CNS tumors (Zhang et al. Int J Cancer (1997); 73:42-49; Dippold et al., Cancer Res (1985); 45:3699-3705; Brezicka et al., Lung Cancer (2000); 28:29-36; Urmacher et al., Am J Dermatopathol (1989); 11:577-581), GD3 expression on normal tissues including skin, adrenal gland, and CNS is generally present at much lower levels compared to neuroendocrine tumors and is mostly cytoplasmic rather than membrane localized (Zhang (1997); Dippold (1985); Urmacher (1989)) outside of the central nervous system (CNS). GD3 is expressed on representative melanoma, SCLC, and sarcoma cell lines, e.g., soft tissue sarcoma (e.g., liposarcoma, and leiomyosarcoma), and bone sarcoma (osteosarcoma) (see FIG. 1A).

### 5.3. Antigen-Recognizing Receptors

The presently disclosed antigen-recognizing receptors specifically target or binds to ganglioside GD3. In certain embodiments, the antigen-recognizing receptor is a chimeric antigen receptor (CAR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a GD3-targeted antigen recognizing receptor disclosed herein.

5.3.1. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to ganglioside GD3, a transmembrane domain, and an intracellular signaling domain.

5.3.1.1. Extracellular Antigen-Binding Domain of A CAR

In certain embodiments, the extracellular antigen-binding domain is an scFv. The scFv can be humanized scFv, murine scFv, or human scFv. In certain embodiments, the scFv is a humanized scFv.

In certain embodiments, the extracellular antigen-binding domain is a Fab. In certain embodiments, the Fab, which is optionally crosslinked. In certain embodiments, the extracellular antigen-binding domain is a F(ab)₂.

In certain embodiments, the extracellular antigen-binding domain of the CAR comprises a humanized scFv. In certain embodiments, the extracellular antigen-binding domain of the CAR (e.g., an scFv) comprises a heavy chain variable region (V_{H}) and a light chain variable region (V_{L}). In certain embodiments, the scFv is a humanized scFv. In certain embodiments, the humanized scFv is derived from R24 antibody, which is a mouse monoclonal antibody that recognizes GD3. R24 antibody has been shown to exhibit a phenomenon known as homophilic binding, whereby the antibodies bind to each other (Yan et al., J Immunol (1996); 157:1582-1588). In certain embodiments, the humanized scFv disclosed herein does not exhibit homophilic binding. In certain embodiments, the V_{H} CDR2 contributes to the absence of homophilic binding. In certain embodiments, the humanized scFv comprises a V_{H} CDR2 comprising or consisting of the amino acid sequence set forth in SEQ ID NO: 2.

In certain embodiments, the V_{H} comprises a CDR1 comprising or consisting of the amino acid sequence set forth in SEQ ID NO: 1 or a conservative modification thereof, a CDR2 comprising or consisting of the amino acid sequence set forth in SEQ ID NO: 2 or a conservative modification thereof, and a CDR3 comprising or consisting of the amino acid sequence set forth in SEQ ID NO: 3 or a conservative modification thereof. SEQ ID NOs: 1-3 are provided in Table 1.

In certain embodiments, the V_{L} comprises a CDR1 comprising or consisting of the amino acid sequence set forth in SEQ ID NO: 4 or a conservative modification thereof, a CDR2 comprising or consisting of the amino acid sequence set forth in SEQ ID NO: 5 or a conservative modification thereof, and a CDR3 comprising or consisting of the amino acid sequence set forth in SEQ ID NO: 6 or a conservative modification thereof. SEQ ID NOs: 4-6 are provided in Table 1.

In certain embodiments, the V_{H} comprises a CDR1 comprising or consisting of the amino acid sequence set forth in SEQ ID NO: 1 or a conservative modification thereof, a CDR2 comprising or consisting of the amino acid sequence set forth in SEQ ID NO: 2 or a conservative modification thereof, and a CDR3 comprising or consisting of the amino acid sequence set forth in SEQ ID NO: 3 or a conservative modification thereof, and the V_{L} comprises a CDR1 comprising or consisting of the amino acid sequence set forth in SEQ ID NO: 4 or a conservative modification thereof, a CDR2 comprising or consisting of the amino acid sequence set forth in SEQ ID NO: 5 or a conservative modification, and a CDR3 comprising or consisting of the amino acid sequence set forth in SEQ ID NO: 6 or a conservative modification thereof.

In certain embodiments, the V_{H} comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the V_{L} comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the CDRs are identified according to the Kabat numbering system.

In certain embodiments, the V_{H} comprises an amino acid sequence that is at least about 80% (e.g., at least about 85%, at least about 90%, or at least about 95%) homologous or identical to the amino acid sequence set forth in SEQ ID NO: 7. For example, the V_{H} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 7. In certain embodiments, the V_{H} comprises the amino acid sequence set forth in SEQ ID NO: 7. SEQ ID NO: 7 is provided in Table 1 below.

In certain embodiments, the V_{L} comprises an amino acid sequence that is at least about 80% (e.g., at least about 85%, at least about 90%, or at least about 95%) homologous or identical to the amino acid sequence set forth in SEQ ID NO: 8. For example, the V_{L} comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the V_{L} comprises the amino acid sequence set forth in SEQ ID NO: 8. In certain embodiments, the V_{L} comprises amino acids 1 to 107 of SEQ ID NO: 8. SEQ ID NO: 8 is provided in Table 1 below.

In certain embodiments, the V_{H} comprises the amino acid sequence set forth in SEQ ID NO: 7, and the V_{L} comprises the amino acid sequence set forth in SEQ ID NO: 8. In certain embodiments, the V_{H} and V_{L} are linked via a linker. In certain embodiments, the linker comprises the amino acid sequence set forth in SEQ ID NO: 18.

In certain embodiments, the variable regions within the extracellular antigen-binding domain of the CAR have to be linked one after another such that at the N-terminus of the extracellular antigen-binding domain, a V_{H} is positioned. In certain embodiments, if the extracellular antigen-binding domain of the CAR is an scFv, the variable regions are positioned from the N- to the C-terminus: V_{H}-V_{L}. In certain embodiments, the scFv comprises the amino acid sequence set forth in SEQ TD NO: 9, which is provided in Table 1. An exemplary nucleotide sequence encoding the amino acid sequence of SEQ TD NO: 9 is set forth in SEQ TD NO: 11, which is provided in Table 1 below.

In certain embodiments, the variable regions within the extracellular antigen-binding domain of the CAR have to be linked one after another such that at the N-terminus of the extracellular antigen-binding domain, a V_{L} is positioned. In certain embodiments, ifthe extracellular antigen-binding domain of the CAR is an scFv, the variable regions are positioned from the N- to the C-terminus: V_{L}-V_{H}. In certain embodiments, scFv comprises the amino acid sequence set forth in SEQ ID NO: 10, which is provided in Table 1 below.

TABLE 1

| Antigen | Ganglioside GD3 | | |
|---|---|---|---|
| CDRs | 1 | 2 | 3 |
| V_{H} | NFGMH [SEQ ID NO: | YITSGGSSINYADTVKG | GGTGTRSLYYFDY |
| | 1] | [SEQ ID NO: 2] | [SEQ ID NO: 3] |
| V_{L} | RASQDIGNELN [SEQ ID | YTSRLQS [SEQ ID NO: | QQGKTLPYT [SEQ |
| | NO: 4] | 5] | ID NO: 6] |

| | |
|---|---|
| Full V_{H} | QVQLVESGGG VVQPGRSLRL SCAASGFTFS NFGMHWVRQA PGKGLEWVAY |
| | ITSGGSSINY ADTVKGRFTI SRDNSKNTLY LQMNSLRAED TAVYYCTRGG |
| | TGTRSLYYFD YWGQGTTVTV SS [SEQ ID NO: 7] |
| Full V_{L} | DIQMTQSPRS LSASVGDRVT ITCRASQDIG NFLNWYQQKP GKAPKLLIYY |
| | TSRLQSGVPS RFSGSGSGTD YTLTISSLQP EDFATYYCQQ GKTLPYTFGG |
| | GTKVEIKD [SEQ ID NO: 8] |
| V_{H}-V_{L} | QVQLVESGGG VVQPGRSLRL SCAASGFTFS NFGMHWVRQA PGKGLEWVAY |
| scFv | ITSGGSSINY ADTVKGRFTI SRDNSKNTLY LQMNSLRAED TAVYYCTRGG |
| | TGTRSLYYFD YWGQGTTVTV SSGGGGSGGG GSGGGGS DIQMTQSPRS |
| | LSASVGDRVT ITCRASQDIG NFLNWYQQKP GKAPKLLIYY TSRLQSGVPS |
| | RFSGSGSGTD YTLTISSLQP EDFATYYCQQ GKTLPYTFGG GTKVEIKD [SEQ |
| | ID NO: 9] |
| V_{L}-V_{H} | DIQMTQSPRS LSASVGDRVT ITCRASQDIG NELNWYQQKP GKAPKLLIYY |
| scFv | TSRLQSGVPS RFSGSGSGTD YTLTISSLQP EDFATYYCQQ GKTLPYTFGG |
| | GTKVEIKD GGGGSGGG GSGGGGS QVQLVESGGG VVQPGRSLRL SCAASGFTES |
| | NFGMHWVRQA PGKGLEWVAY ITSGGSSINY ADTVKGRFTI SRDNSKNTLY |
| | LQMNSLRAED TAVYYCTRGG TGTRSLYYFD YWGQGTTVTV SS [SEQ ID NO: |
| | 10] |
| DNA for | CAGGTGCAGCTGGTGGAGAGCGGCGGCGGCGTGGTGCAGCCCGGCAGAAGCCTG |
| V_{H}-V_{L} | AGACTGAGCTGCGCCGCCAGCGGCTTCACCTTCAGCAACTTCGGCATGCACTGG |
| scFv | GTGAGACAGGCCCCCGGCAAGGGCCTGGAGTGGGTGGCCTACATCACCAGCGGC |
| | GGCAGCAGCATCAACTACGCCGACACCGTGAAGGGCAGATTCACCATCAGCAGA |
| | GACAACAGCAAGAACACCCTGTACCTGCAGATGAACAGCCTGAGAGCCGAGGAC |
| | ACCGCCGTGTACTACTGCACCAGAGGCGGCACCGGCACCAGAAGCCTGTACTAC |
| | TTCGACTACTGGGGCCAGGGCACCACCGTGACCGTGAGCAGCGGTGGAGGTGGA |
| | TCAGGTGGAGGTGGATCTGGTGGAGGTGGATCTGACATCCAGATGACCCAGAGC |
| | CCCAGAAGCCTGAGCGCCAGCGTGGGCGACAGAGTGACCATCACCTGCAGAGCC |
| | AGCCAGGACATCGGCAACTTCCTGAACTGGTACCAACAGAAGCCCGGCAAGGCC |
| | CCCAAGCTGCTGATCTACTACACCAGTAGACTGCAGAGCGGCGTGCCCAGCAGA |
| | TTTAGTGGTAGCGGTAGCGGCACCGACTACACCCTGACCATCAGCAGCCTGCAG |
| | CCCGAGGACTTCGCCACCTACTACTGCCAGCAGGGCAAGACCCTGCCCTACACC |
| | TTCGGCGGCGGCACCAAGGTGGAGATCAAGGAC [SEQ ID NO: 11] |

In certain embodiments, the V_{H} comprises a CDR1 comprising or consisting of the amino acid sequence set forth in SEQ ID NO: 1 or a conservative modification thereof, a CDR2 comprising or consisting of the amino acid sequence set forth in SEQ ID NO: 12 or a conservative modification thereof, and a CDR3 comprising or consisting of the amino acid sequence set forth in SEQ ID NO: 3 or a conservative modification thereof. SEQ ID NO: 12 is provided below.

[SEQ ID NO: 12]
YISSGGSSINYADTV

In certain embodiments, the V_{H} comprises a CDR1 comprising or consisting of the amino acid sequence set forth in SEQ ID NO: 1 or a conservative modification thereof, a CDR2 comprising or consisting of the amino acid sequence set forth in SEQ ID NO: 12 or a conservative modification thereof, and a CDR3 comprising or consisting of the amino acid sequence set forth in SEQ ID NO: 3 or a conservative modification thereof, and the V_{L} comprises a CDR1 comprising or consisting of the amino acid sequence set forth in SEQ ID NO: 4 or a conservative modification thereof, a CDR2 comprising or consisting of the amino acid sequence set forth in SEQ ID NO: 5 or a conservative modification, and a CDR3 comprising or consisting of the amino acid sequence set forth in SEQ ID NO: 6 or a conservative modification thereof.

In certain embodiments, the V_{H} comprises a CDR1 comprising the amino acid sequence set forth in SEQ ID NO: 1, a CDR2 comprising the amino acid sequence set forth in SEQ ID NO: 12, and a CDR3 comprising the amino acid sequence set forth in SEQ ID NO: 3; and the V_{L} comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the V_{H} comprises the amino acid sequence set forth in SEQ ID NO: 13, and the V_{L} comprises the amino acid sequence set forth in SEQ ID NO: 14. In certain embodiments, the V_{H} and V_{L} are linked via a linker. SEQ ID Nos: 13 and 14 are provided below. In certain embodiments, the extracellular antigen-binding domain comprises the V_{H} and V_{L} CDRs sequences of humanized antibody “LD47” disclosed in U.S. Pat. Nos. 8,207,308 and 9,205,157, which are incorporated by reference herein by their entirety. In certain embodiments, the extracellular antigen-binding domain comprises the V_{H} and V_{L} sequences of humanized antibody “LD47” disclosed in U.S. Pat. Nos. 8,207,308 and 9,205,157.

[SEQ ID NO: 13]
QVQLVESGGGVVQPGRSLRLSCAASGFTFSNFGMHWVRQAPGKGLEWVA
YISSGGSSINYADTVKGRFTISRDNSKNTLYLQMNSLRAEDTAVYYCTR
GGTGTRSLYYFDYWGQGTTVTVSS
[SEQ ID NO: 14]
DIQMTQSPSSLSASVGDRVTITCRASQDIGNFLNWYQQKPGKAPKLLIY
YTSRLQSGVPSRFSGSGSGTDYTLTISSLQPEDFATYYCQQGKTLPYTF
GGGTKVEIK

In certain embodiments, the V_{H} comprises the amino acid sequence set forth in SEQ ID NO: 15, and the V_{L} comprises the amino acid sequence set forth in SEQ ID NO: 16. In certain embodiments, the V_{H} and V_{L} are linked via a linker. SEQ ID Nos: 15 and 16 are provided below. In certain embodiments, the extracellular antigen-binding domain comprises the V_{H} and V_{L} CDRs sequences of humanized antibody “LD49” disclosed in U.S. Pat. Nos. 8,207,308 and 9,205,157, which are incorporated by reference herein by their entirety. In certain embodiments, the extracellular antigen-binding domain comprises the V_{H} and V_{L} sequences of humanized antibody “LD49” disclosed in U.S. Pat. Nos. 8,207,308 and 9,205,157.

[SEQ ID NO: 15]
DVQLVESGGGLVQPGGSRKLSCAASGFTFSNFGMHWVRQAPEKGLEWVA
YISSGGSSINYADTVKGRFTISRDNPKNTLFLOMTSLRSEDTAIYYCTR
GGTGTRSLYYFDYWGOGATLIVSS
[SEQ ID NO: 16]
DIQMTQSPSSLSASVGDRVTITCRASQDIGNFLNWYQQKPGKAPKLLLY
YTSRLQSGVPSRESGSGSGTDYTLTISSLQPEDFATYYCQQGKTLPYTF
GGGTKVEIK

As used herein, the term “a conservative sequence modification” refers to an amino acid modification that does not significantly affect or alter the binding characteristics of the presently disclosed mesothelin-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences comprising or consisting of at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 7 or SEQ ID NO: 8) may contain substitutions (e.g., conservative substitutions), insertions, or deletions relative to the specified sequence(s), but retain the ability to bind to a target antigen (e.g., mesothelin). In certain embodiments, a total of 1 to 10 amino acids are substituted, inserted and/or deleted in a specific sequence (e.g., SEQ ID NO: 7 or SEQ ID NO: 8). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 7 or SEQ ID NO: 8, including post-translational modifications of that sequence (SEQ ID NO: 7 or SEQ ID NO: 8).

In certain embodiments, the extracellular antigen-binding domain of a presently disclosed CAR cross-competes for binding to ganglioside GD3 with a reference antibody or an antigen-binding portion thereof comprising the a V_{H} CDR1 comprising the amino acid sequence set forth in SEQ ID NO: 1, a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR cross-competes for binding to ganglioside GD3 with a reference antibody or an antigen-binding portion thereof comprising a V_{H} comprising the amino acid sequence set forth in SEQ ID NO: 7, and a V_{L} comprising the amino acid sequence set forth in SEQ ID NO: 8.

In certain embodiments, the extracellular antigen-binding domain of a presently disclosed CAR cross-competes for binding to ganglioside GD3 with a reference antibody or an antigen-binding portion thereof comprising the a V_{H} CDR1 comprising the amino acid sequence set forth in SEQ ID NO: 1, a V_{H} CDR2 comprising the amino acid sequence set forth in SEQ ID NO: 12; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR cross-competes for binding to ganglioside GD3 with a reference antibody or an antigen-binding portion thereof comprising a V_{H} comprising the amino acid sequence set forth in SEQ ID NO: 13, and a V_{L} comprising the amino acid sequence set forth in SEQ ID NO: 14. In certain embodiments, the extracellular antigen-binding domain of a presently disclosed CAR cross-competes for binding to ganglioside GD3 with a reference antibody or an antigen-binding portion thereof comprising a V_{H} comprising the amino acid sequence set forth in SEQ ID NO: 15, and a V_{L} comprising the amino acid sequence set forth in SEQ ID NO: 16.

In certain embodiments, the extracellular antigen-binding domain binds to the same or substantially the same epitope on ganglioside GD3 as the reference antibody or antigen-binding portion thereof comprising a V_{H} CDR1 comprising the amino acid sequence set forth in SEQ ID NO: 1; a V_{H} CDR2 comprising the amino acid sequence set forth in SEQ ID NO: 2; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR binds to the same or substantially the same epitope on ganglioside GD3 as a reference antibody or an antigen-binding fragment thereof comprising a V_{H} comprising the amino acid sequence set forth in SEQ ID NO: 7, and a V_{L} comprising the amino acid sequence set forth in SEQ ID NO: 8.

In certain embodiments, the extracellular antigen-binding domain binds to the same or substantially the same epitope on ganglioside GD3 as the reference antibody or antigen-binding portion thereof comprising a V_{H} CDR1 comprising the amino acid sequence set forth in SEQ ID NO: 1; a V_{H} CDR2 comprising the amino acid sequence set forth in SEQ ID NO: 12; a V_{H} CDR3 comprising the amino acid sequence set forth in SEQ ID NO: 3; a V_{L} CDR1 comprising the amino acid sequence set forth in SEQ ID NO: 4; a V_{L} CDR2 comprising the amino acid sequence set forth in SEQ ID NO: 5; and a V_{L} CDR3 comprising the amino acid sequence set forth in SEQ ID NO: 6. In certain embodiments, the extracellular antigen-binding domain of a presently disclosed CAR binds to the same or substantially the same epitope on ganglioside GD3 as a reference antibody or an antigen-binding fragment thereof comprising a V_{H} comprising the amino acid sequence set forth in SEQ ID NO: 13, and a V_{L} comprising the amino acid sequence set forth in SEQ ID NO: 14. In certain embodiments, the extracellular antigen-binding domain of a presently disclosed CAR binds to the same or substantially the same epitope on ganglioside GD3 as a reference antibody or an antigen-binding fragment thereof comprising a V_{H} comprising the amino acid sequence set forth in SEQ ID NO: 15, and a V_{L} comprising the amino acid sequence set forth in SEQ ID NO: 16.

Extracellular antigen-binding domains that cross-compete or compete with the reference antibody or antigen-binding portions thereof for binding to ganglioside GD3 can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a ganglioside GD3, admixing the reference antibody with a test extracellular antigen-binding domain, measuring a second binding of the reference antibody to the ganglioside GD3 in the presence of the test extracellular antigen-binding domain, and comparing the initial binding with the second binding of the reference antibody, wherein a decreased second binding of the reference antibody to the ganglioside GD3 in comparison to the initial binding indicates that the test extracellular antigen-binding domain cross-competes with the reference antibody for binding to ganglioside GD3, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the ganglioside GD3 is expressed in cells, e.g., in a flow cytometry test. In certain embodiments, the ganglioside GD3 is immobilized onto a surface, including a Biacore ship (e.g., in a Biacore test), or other media suitable for surface plasmon resonance analysis. The binding of the reference antibody in the presence of a completely irrelevant antibody (that does not bind to ganglioside GD3) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to ganglioside GD3 by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to ganglioside GD3. In certain embodiments, the assays are performed at room temperature.

In certain embodiments, the antibody-binding assay comprises measuring an initial binding of a test extracellular antigen-binding domain to ganglioside GD3, admixing the test extracellular antigen-binding domain with a reference antibody, measuring a second binding of the test extracellular antigen-binding domain to ganglioside GD3 in the presence of the reference antibody, and comparing the initial binding with the second binding of the test extracellular antigen-binding domain, where a decreased second binding of the test extracellular antigen-binding domain to ganglioside GD3 in comparison to the initial binding indicates that the test extracellular antigen-binding domain cross-competes with the reference antibody for binding to ganglioside GD3, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the ganglioside GD3 is expressed in cells, e.g., in a flow cytometry test. In certain embodiments, the ganglioside GD3 is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to ganglioside GD3) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to ganglioside GD3 is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to ganglioside GD3. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises the amino acid sequence set forth in SEQ ID NO: 17. In certain embodiments, the linker comprises the amino acid sequence set forth in SEQ ID NO: 18. In certain embodiments, the linker comprises the amino acid sequence set forth in SEQ ID NO: 19. In certain embodiments, the linker comprises the amino acid sequence set forth in SEQ ID NO: 20. In certain embodiments, the linker comprises the amino acid sequence set forth in SEQ ID NO: 21. In certain embodiments, the linker comprises the amino acid sequence set forth in SEQ ID NO: 22.

In certain embodiments, the variable regions within the extracellular antigen-binding domain of the CAR have to be linked one after another such that at the N-terminus of the extracellular antigen-binding domain, a heavy chain variable region (V_{H}) is positioned. In certain embodiments, if the extracellular antigen-binding domain of the CAR is an scFv, the variable regions are positioned from the N- to the C-terminus: V_{H}-V_{L}.

In certain embodiments, the variable regions within the extracellular antigen-binding domain of the CAR have to be linked one after another such that at the N-terminus of the extracellular antigen-binding domain, a light chain variable region (V_{L}) is positioned. In certain embodiments, if the extracellular antigen-binding domain of the CAR is an scFv, the variable regions are positioned from the N- to the C-terminus: V_{L}-V_{H}.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide.

5.3.1.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof).

In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 23) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 23, which is at least 20, or at least 30, or at least 40, or at least 50, and up to 235 amino acids in length. In certain embodiments, the CD8 polypeptide comprises or consists of amino acids 1 to 235, 1 to 50, 50 to 100, 100 to 150, 150 to 200, 137 to 209 or 200 to 235 of SEQ ID NO: 23. In certain embodiments, the transmembrane domain of the CAR comprises a CD8 polypeptide comprising or consisting of amino acids 137 to 209 of SEQ ID NO: 23. SEQ ID NO: 23 is provided below.

[SEQ ID NO: 23]
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDERRENEGYYFCSALSNSIMYFSHFVPVELPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV

In certain embodiments, the transmembrane domain of the CAR comprises a CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 24) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 24, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of amino acids 1 to 247, 1 to 50, 50 to 100, 100 to 150, 150 to 200, 151 to 219, or 200 to 247 of SEQ ID NO: 24. In certain embodiments, the transmembrane domain of the CAR comprises a CD8 polypeptide comprising or consisting of amino acids 151 to 219 of SEQ ID NO: 24. SEQ ID NO: 24 is provided below.

| [SEQ ID NO: 24] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPOPTFV VYMASSHNKI TWDEKLNSSK LFSAVRDTNN KYVLTLNKES |
| 121 | KENEGYYFCS VISNSVMYFS SVVPVLQKVN STTTKPVLRT PSPVHPTGTS QPORPEDCRP |
| 181 | RGSVKGTGLD FACDIYIWAP LAGICVAPLL SLIITLICYH RSRKRVCKCP RPLVROEGKP |
| 241 | RPSEKIV |

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID NO: 25) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 25, which is at least 20, or at least 30, or at least 40, or at least 50, and up to 220 amino acids in length. In certain embodiments, the CD28 polypeptide comprises or consists of amino acids 1 to 220, 1 to 50, 50 to 100, 100 to 150, 150 to 200, 153 to 179, or 200 to 220 of SEQ ID NO: 25. In certain embodiments, the transmembrane domain of the CAR comprises a CD28 polypeptide comprising or consisting of amino acids 153 to 179 of SEQ ID NO: 25. SEQ ID NO: 25 is provided below:

| [SEQ ID NO: 25] | |
|---|---|
| 1 | MLRLLLALNL FPSIQVTGNK ILVKOSPMLV AYDNAVNLSC KYSYNLESRE FRASLHKGLD |
| 61 | SAVEVCVVYG NYSQQLQVYS KTGENCDGKL GNESVTFYLQ NLYVNOTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acid 153 to 179 of SEQ ID NO: 25 is set forth in SEQ ID NO: 26, which is provided below.

[SEQ ID NO: 26]
TTTTGGGTGCTGGTGGTGGTTGGTGGAGTCCTGGCTTGCTATAGCTTGC
TAGTAACAGTGGCCTTTATTATTTTCTGGGTG

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID NO: 27) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 27, which is at least 20, or at least 30, or at least 40, or at least 50, and up to 218 amino acids in length. In certain embodiments, the CD28 polypeptide comprises or consists of amino acids 1 to 220, 1 to 50, 50 to 100, 100 to 150, 150 to 200, 151 to 177, or 200 to 218 of SEQ ID NO: 27. In certain embodiments, the transmembrane domain of the CAR comprises a CD28 polypeptide comprising or consisting of amino acids 151 to 177 of SEQ ID NO: 27. SEQ ID NO: 27 is provided below:

| [SEQ ID NO: 27] | |
|---|---|
| 1 | MTLRLLFLAL NFFSVOVTEN KILVKOSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLOSDYM NMTPRRPGLT RKPYQPYAPA RDFAAYRP |

In certain embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 20 or 22), a portion of a CD8 polypeptide (e.g., a portion of SEQ ID NO: 18 or 19), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a CD28 polypeptide. In certain embodiments, the hinge/spacer region of the CAR comprises a CD28 polypeptide comprising or consisting of amino acids 114 to 152 of SEQ ID NO: 25.

An exemplary nucleotide sequence encoding amino acid 114 to 152 of SEQ ID NO: 25 is set forth in SEQ ID NO: 28, which is provided below.

[SEQ ID NO: 28]
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCC

5.3.2.3. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 29) or a fragment thereof, and/or may optionally comprise up to one or up to two or up to three conservative amino acid substitutions. In certain embodiments, the native CD3ζ polypeptide comprises or consists of an amino acid sequence that is a consecutive portion of SEQ ID NO: 29, which is at least 20, or at least 30, or at least 40, or at least 50, and up to 164 amino acids in length. In certain embodiments, the native CD3ζ polypeptide comprises or consists of amino acids 1 to 164, 1 to 50, 50 to 100, 52 to 164, 100 to 150, or 150 to 164 of SEQ ID NO: 29. In certain embodiments, the intracellular signaling domain of the CAR comprises a CD3ζ polypeptide comprising or consisting of amino acids 52 to 164 of SEQ ID NO: 29. SEQ ID NO: 29 is provided below:

| [SEQ ID NO: 29] | |
|---|---|
| 1 | MKWKALFTAA ILQAQLPITE AQSFGLLDPK LCYLLDGILF IYGVILTALF LRVKFSRSAD |
| 61 | APAYQQGONQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a CD3ζ polypeptide comprising or consisting of the amino acid sequence set forth in SEQ ID NO: 30, which is provided below.

[SEQ ID NO: 30]
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR

An exemplary nucleotide sequence encoding the amino acid sequence of SEQ ID NO: 30 is set forth in SEQ ID NO: 31, which is as provided below.

[SEQ ID NO: 31]
AGAGTGAAGTTCAGCAGGAGCGCAGACGCCCCCGCGTACCAGCAGGGCCAGAACCAGCTCTATAACGAGCTCAATCTAGG
ACGAAGAGAGGAGTACGATGTTTTGGACAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACAGTGAGATTGGGATGAAAGGCGAGCGC
CGGAGGGGCAAGGGGCACGATGGCCTTTACCAGGGTCTCAGTACAGCCACCAAGGACACCTACGACGCCCTTCACATGCA
GGCCCTGCCCCCTCGCTAA

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 32.

[SEQ ID NO: 32]
QNQLYNELNLGRREEYDVLDKR

An exemplary nucleotide sequence encoding the amino acid sequence of SEQ ID NO: 32 is set forth in SEQ ID NO: 33, which is provided below.

[SEQ ID NO: 33]
CAGAACCAGCTCTATAACGAGCTCAATCTAGGACGAAGAGAGGAGT
ACGATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 34, which is provided below.

[SEQ ID NO: 34]
QNQLFNELNLGRREEFDVLDKR

An exemplary nucleotide sequence encoding the amino acid sequence of SEQ ID NO: 34 is set forth in SEQ ID NO: 35, which is provided below.

[SEQ ID NO: 35]
CAGAACCAGCTCTTTAACGAGCTCAATCTAGGACGAAGAG
AGGAGTTCGATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 36, which is provided below.

[SEQ ID NO: 36]
QEGLYNELQKDKMAEAYSEIGMK

An exemplary nucleotide sequence encoding the amino acid sequence of SEQ ID NO: 36 is set forth in SEQ ID NO: 37, which is provided below.

[SEQ ID NO: 37]
CAGGAAGGCCTGTACAATGAACTGCAGAAAGATAAGATGG
CGGAGGCCTACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 38, which is provided below.

[SEQ ID NO: 38]
QEGLENELOKDKMAEAFSEIGMK

An exemplary nucleotide sequence encoding the amino acid sequence of SEQ ID NO: 38 is set forth in SEQ ID NO: 39, which is provided below.

[SEQ ID NO: 39]
CAGGAAGGCCTGTTCAATGAACTGCAGAAAGATAAGATGG
CGGAGGCCTTCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 40, which is provided below.

[SEQ ID NO: 40]
HDGLYQGLSTATKDTYDALHMQ

An exemplary nucleotide sequence encoding the amino acid sequence of SEQ ID NO: 40 is set forth in SEQ ID NO: 41, which is provided below.

[SEQ ID NO: 41]
CACGATGGCCTTTACCAGGGTCTCAGTACAGCCACCAAGG
ACACCTACGACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 42, which is provided below.

[SEQ ID NO: 42]
HDGLFQGLSTATKDTFDALHMQ

An exemplary nucleotide sequence encoding the amino acid sequence of SEQ ID NO: 42 is set forth in SEQ ID NO: 43, which is provided below.

[SEQ ID NO: 43]
CACGATGGCCTTTTCCAGGGGCTCAGTACAGCCACCAAGG
ACACCTTCGACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 32, an ITAM2 variant consisting of the amino acid sequence set forth in SEQ ID NO: 38, and an ITAM3 variant consisting of the amino acid sequence set forth in SEQ ID NO: 42. In certain embodiments, the modified CD3ζ polypeptide is designated as “1XX”. In certain embodiments, the modified CD3ζ polypeptide comprises or consists of the amino acid sequence set forth in SEQ ID NO: 44. SEQ ID NO: 44 is provided below.

[SEQ ID NO: 44]
RVKFSRSADA PAYQQGQNQL YNELNLGRRE EYDVLDK
RRGRDPEMGGKPR RKNPQEGLEN ELQKDKMAEA FSEI
GMKGERRRGKGHDGLF QGLSTATKDT FDALHMQALP P
R

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 44 or a fragment thereof, and/or may optionally comprise up to one or up to two or up to three conservative amino acid substitutions.

An exemplary nucleotide sequence encoding the amino acid sequence of SEQ ID NO: 44 is set forth in SEQ ID NO: 45, which is provided below.

[SEQ ID NO: 45]
AGAGTGAAGTTCAGCAGGAGCGCAGACGCCCCCGCGTACC
AGCAGGGCCAGAACCAGCTCTATAACGAGCTCAATCTAGG
ACGAAGAGAGGAGTACGATGTTTTGGACAAGAGACGTGGC
CGGGACCCTGAGATGGGGGGAAAGCCGAGAAGGAAGAACC
CTCAGGAAGGCCTGTTCAATGAACTGCAGAAAGATAAGAT
GGCGGAGGCCTTCAGTGAGATTGGGATGAAAGGCGAGCGC
CGGAGGGGCAAGGGGCACGATGGCCTTTTCCAGGGGCTCA
GTACAGCCACCAAGGACACCTTCGACGCCCTTCACATGCA
GGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. The CD28 polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 25 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 25, which is at least 20, or at least 30, or at least 40, or at least 50, and up to 220 amino acids in length. In certain embodiments, the CD28 polypeptide comprises or consists of amino acids 1 to 220, 1 to 50, 50 to 100, 100 to 150, 114 to 220, 150 to 200, 180 to 220, or 200 to 220 of SEQ ID NO: 25. In certain embodiments, the intracellular signaling domain of the CAR comprises a co-stimulatory signaling region that comprises a CD28 polypeptide comprising or consisting of amino acids 180 to 220 of SEQ ID NO: 25.

An exemplary nucleic acid sequence encoding amino acids 180 to 220 of SEQ ID NO: 25 is set forth in SEQ ID NO: 46, which is provided below.

[SEQ ID NO: 46]
AGGAGTAAGAGGAGCAGGCTCCTGCACAGTGACTACATGA
ACATGACTCCCCGCCGCCCCGGGCCCACCCGCAAGCATTA
CCAGCCCTATGCCCCACCACGCGACTTCGCAGCCTATCGC
TCC

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 27 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 27, which is at least about 20, or at least about 30, or at least about 40, or at least about 50, and up to 218 amino acids in length. In certain embodiments, the CD28 polypeptide comprises or consists of amino acids 1 to 218, 1 to 50, 50 to 100, 100 to 150, 150 to 218, 178 to 218, or 200 to 218 of SEQ ID NO: 27. In certain embodiments, the co-stimulatory signaling region of a presently disclosed CAR comprises a CD28 polypeptide that comprises or consists of the amino acids 178 to 218 of SEQ ID NO: 27.

In certain embodiments, the intracellular signaling domain of the CAR comprises a co-stimulatory signaling region that comprises a 4-1BB polypeptide, e.g., an intracellular domain of 4-1BB or a fragment thereof (e.g., an intracellular domain of human 4-1BB or a fragment thereof). The 4-1BB polypeptide can comprise or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 47) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 47, which is at least 20, or at least 30, or at least 40, or at least 50, or at least 100, or at least 150, or at least 150, and up to 255 amino acids in length. In certain embodiments, the 4-1BB polypeptide comprises or consists of amino acids 1 to 255, 1 to 50, 50 to 100, 100 to 150, 150 to 200, or 200 to 255 of SEQ ID NO: 47. In certain embodiments, the intracellular signaling domain of the CAR comprises a co-stimulatory signaling region that comprises a 4-1BB polypeptide comprising or consisting of amino acids 214 to 255 of SEQ ID NO: 47. SEQ ID NO: 47 is provided below.

[SEQ ID NO: 47]

| | |
|---|---|
| 1 | MGNSCYNIVA TLLLVLNFER TRSLQDPCSN |
| | CPAGTFCDNN RNQICSPCPP NSESSAGGQR |
| 61 | TCDICRQCKG VERTRKECSS TSNAECDCTP |
| | GFHCLGAGCS MCEQDCKQGQ ELTKKGCKDC |
| 121 | CFGTENDQKR GICRPWTNCS LDGKSVLVNG |
| | TKERDVVCGP SPADLSPGAS SVTPPAPARE |
| 181 | PGHSPQIISF FLALTSTALL FLLFFLTLRF |
| | SVVKRGRKKL LYIFKQPFMR PVQTTQEEDG |
| 241 | CSCRFPEEEE GGCEL |

An exemplary nucleic acid sequence encoding amino acids 214 to 255 of SEQ ID NO: 47 is set forth in SEQ ID NO: 48, which is provided below.

[SEQ ID NO: 48]
AAACGGGGCAGAAAGAAACTCCTGTATATATTCAAACAAC
CATTTATGAGACCAGTACAAACTACTCAAGAGGAAGATGG
CTGTAGCTGCCGATTTCCAGAAGAAGAAGAAGGAGGATGT
GAACTG

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

5.3.1.4. Exemplified CARs

In certain embodiments, the CAR is a GD3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a V_{H} CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a V_{L} CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 18. In certain embodiments, the V_{H} and V_{L} are positioned from the N- to the C-terminus: V_{H}-V_{L}. In certain embodiments, the CAR is designed as “hR24v”. In certain embodiments, the CAR comprises the amino acid sequence set forth in SE ID NO: 49, which is provided below.

[SEQ ID NO: 49]
MALPVTALLLPLALLLHAEQVQLVESGGGVVQPGRSLRLS
CAASGFTFSNFGMHWVRQAPGKGLEWVAYITSGGSSINYA
DTVKGRFTISRDNSKNTLYLQMNSLRAEDTAVYYCTRGGT
GTRSLYYFDYWGQGTTVTVSSGGGGSGGGGSGGGGSDIQM
TQSPRSLSASVGDRVTITCRASQDIGNFLNWYQQKPGKAP
KLLIYYTSRLQSGVPSRFSGSGSGTDYTLTISSLQPEDFA
TYYCQQGKTLPYTFGGGTKVEIKAAAAAIEVMYPPPYLDN
EKSNGTIIHVKGKHLCPSPLFPGPSKPFWVLVVVGGVLAC
YSLLVTVAFIIFWVRSKRSRLLHSDYMNMTPRRPGPTRKH
YQPYAPPRDFAAYRSRVKFSRSADAPAYQQGQNQLYNELN
LGRREEYDVLDKRRGRDPEMGGKPRRKNPQEGLYNELQKD
KMAEAYSEIGMKGERRRGKGHDGLYQGLSTATKDTYDALH
MQALPPR

An exemplary nucleic acid sequence the amino acid sequence of SEQ ID NO: 49 is set forth in SEQ ID NO: 50, which is provided below.

SEQ ID NO: 50]
ATGGCTCTCCCAGTGACTGCCCTACTGCTTCCCCTAGCGCTTCTCCTGCATGCTGAACAGGTGCAGCTGGTGGAGAGCGG
CGGCGGCGTGGTGCAGCCCGGCAGAAGCCTGAGACTGAGCTGCGCCGCCAGCGGCTTCACCTTCAGCAACTTCGGCATGC
ACTGGGTGAGACAGGCCCCCGGCAAGGGCCTGGAGTGGGTGGCCTACATCACCAGCGGCGGCAGCAGCATCAACTACGCC
GACACCGTGAAGGGCAGATTCACCATCAGCAGAGACAACAGCAAGAACACCCTGTACCTGCAGATGAACAGCCTGAGAGC
CGAGGACACCGCCGTGTACTACTGCACCAGAGGCGGCACCGGCACCAGAAGCCTGTACTACTTCGACTACTGGGGCCAGG
GCACCACCGTGACCGTGAGCAGCGGTGGAGGTGGATCAGGTGGAGGTGGATCTGGTGGAGGTGGATCTGACATCCAGATG
ACCCAGAGCCCCAGAAGCCTGAGCGCCAGCGTGGGCGACAGAGTGACCATCACCTGCAGAGCCAGCCAGGACATCGGCAA
CTTCCTGAACTGGTACCAACAGAAGCCCGGCAAGGCCCCCAAGCTGCTGATCTACTACACCAGTAGACTGCAGAGCGGCG
TGCCCAGCAGATTTAGTGGTAGCGGTAGCGGCACCGACTACACCCTGACCATCAGCAGCCTGCAGCCCGAGGACTTCGCC
ACCTACTACTGCCAGCAGGGCAAGACCCTGCCCTACACCTTCGGCGGCGGCACCAAGGTGGAGATCAAGGACGCGGCCGC
ggccGCAATTGAAGTTATGTATCCTCCTCCTTACCTAGACAATGAGAAGAGCAATGGAACCATTATCCATGTGAAAGGGA
AACACCTTTGTCCAAGTCCCCTATTTCCCGGACCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAGGAGTAAGAGGAGCAGGCTCCTGCACAGTGACTA
CATGAACATGACTCCCCGCCGCCCCGGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGATGGGGGGAAAGCCGAGAAG
GAAGAACCCTCAGGAAGGCCTGTACAATGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTACAGCCACCAAGGACACCTACGACGCCCTT
CACATGCAGGCCCTGCCCCCTCGCTAA

In certain embodiments, the CAR comprises a FLAG epitope tag, which has the amino acid sequence set forth in SEQ ID NO: 51, which is provided below.

[SEQ ID NO: 51]
DYKDDDDK

In certain embodiments, the FLAG epitope tag is position between the extracellular antigen-binding domain and the transmembrane domain. In certain embodiments, the CAR comprising the FLAG epitope tag comprises the amino acid sequence set forth in SEQ ID NO: 52, which is provided below.

[SEQ ID NO: 52]
MALPVTALLLPLALLLHAEQVOLVESGGGVVQPGRSLRLSCAASGFTFSNFGMHWVRQAPGKGLEWVAYITSGG
SSINYADTVKGRFTISRDNSKNTLYLQMNSLRAEDTAVYYCTRGGTGTRSLYYFDYWGQGTTVTVSSGGGGSGGGGSGGG
GSDIQMTQSPRSLSASVGDRVTITCRASQDIGNFLNWYQQKPGKAPKLLIYYTSRLQSGVPSRFSGSGSGTDYTLTISSL
QPEDFATYYCQQGKTLPYTFGGGTKVEIKDDYKDDDDKAAAAAIEVMYPPPYLDNEKSNGTIIHVKGKHLCPSPLFPGPS
KPFWVLVVVGGVLACYSLLVTVAFIIFWVRSKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYNELQKDKMAEAYSEIGMKGERRRGKGHDGLY
QGLSTATKDTYDALHMQALPPR

An exemplary nucleic acid sequence the amino acid sequence of SEQ ID NO: 52 is set forth in SEQ ID NO: 53, which is provided below.

[SEQ ID NO: 53]
ATGGCTCTCCCAGTGACTGCCCTACTGCTTCCCCTAGCGCTTCTCCTGCATGCTGAACAGGTGCAGCTGGTGGAGAGCGG
CGGCGGCGTGGTGCAGCCCGGCAGAAGCCTGAGACTGAGCTGCGCCGCCAGCGGCTTCACCTTCAGCAACTTCGGCATGC
ACTGGGTGAGACAGGCCCCCGGCAAGGGCCTGGAGTGGGTGGCCTACATCACCAGCGGCGGCAGCAGCATCAACTACGCC
GACACCGTGAAGGGCAGATTCACCATCAGCAGAGACAACAGCAAGAACACCCTGTACCTGCAGATGAACAGCCTGAGAGC
CGAGGACACCGCCGTGTACTACTGCACCAGAGGCGGCACCGGCACCAGAAGCCTGTACTACTTCGACTACTGGGGCCAGG
GCACCACCGTGACCGTGAGCAGCGGTGGAGGTGGATCAGGTGGAGGTGGATCTGGTGGAGGTGGATCTGACATCCAGATG
ACCCAGAGCCCCAGAAGCCTGAGCGCCAGCGTGGGCGACAGAGTGACCATCACCTGCAGAGCCAGCCAGGACATCGGCAA
CTTCCTGAACTGGTACCAACAGAAGCCCGGCAAGGCCCCCAAGCTGCTGATCTACTACACCAGTAGACTGCAGAGCGGCG
TGCCCAGCAGATTTAGTGGTAGCGGTAGCGGCACCGACTACACCCTGACCATCAGCAGCCTGCAGCCCGAGGACTTCGCC
ACCTACTACTGCCAGCAGGGCAAGACCCTGCCCTACACCTTCGGCGGCGGCACCAAGGTGGAGATCAAGGACGACTACAA
AGACGATGACGACAAGGCGGCCGCggccGCAATTGAAGTTATGTATCCTCCTCCTTACCTAGACAATGAGAAGAGCAATG
GAACCATTATCCATGTGAAAGGGAAACACCTTTGTCCAAGTCCCCTATTTCCCGGACCTTCTAAGCCCTTTTGGGTGCTG
GTGGTGGTTGGTGGAGTCCTGGCTTGCTATAGCTTGCTAGTAACAGTGGCCTTTATTATTTTCTGGGTGAGGAGTAAGAG
GAGCAGGCTCCTGCACAGTGACTACATGAACATGACTCCCCGCCGCCCCGGGCCCACCCGCAAGCATTACCAGCCCTATG
CCCCACCACGCGACTTCGCAGCCTATCGCTCCAGAGTGAAGTTCAGCAGGAGCGCAGACGCCCCCGCGTACCAGCAGGGC
CAGAACCAGCTCTATAACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGATGGCCTTTACCAGGGTCTCAGTACAGCC
ACCAAGGACACCTACGACGCCCTTCACATGCAGGCCCTGCCCCCTCGCTAA

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

5.3.2. TCR like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5.4. Cells

The presently disclosed subject matter provides cells comprising a presently disclosed GD3-targeted antigen-recognizing receptor (e.g., one disclosed in Section 5.3). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and T6 T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR like fusion molecule. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed GD3-targeted antigen-recognizing receptor such that the cells express the antigen-recognizing receptor.

Secretin Antibody, Antigen-Binding Fragments, or Fusion Proteins

In certain embodiments, the cell further comprises at least one soluble antibody, a soluble antigen-binding fragment, or a fusion protein, which binds to a polypeptide having immunosuppressive activity or immunostimulatory activity. The soluble antibody, antigen-binding fragment, or fusion protein can enhance the immune response of the cell. In certain embodiments, the soluble antibody, antigen-binding fragment, or fusion protein is secreted from the cell. Non-limiting examples of antibodies, antigen-binding fragments, and fusion proteins include monoclonal antibodies, single-chain variable fragments (scFvs), scFv-Fc fusion proteins, bispecific antibodies, minibodies, and BiTEs. In certain embodiments, the antigen-binding fragment is a scFv. In certain embodiments, the fusion protein is a scFv-Fc fusion protein.

In certain embodiments, the at least one soluble antibody, antigen-binding fragment, or fusion protein binds to a polypeptide having immunosuppressive activity. In certain embodiments, the polypeptide having immunosuppressive activity is selected from CD47, PD-1, CTLA-4, BTLA, LAG-3, 2B4, CD47, and their corresponding ligands or receptors (including, not limited to, SIRPα, PD-L1, PD-L2, TNFRSF14, CD48, and FGL-1). In certain embodiments, the soluble antibody, antigen-binding fragment, or fusion protein is an antagonist antibody, antigen-binding fragment, or fusion protein. In certain embodiments, the soluble antibody, antigen-binding fragment, or fusion protein is an antagonist antibody, antigen-binding fragment, or fusion protein that binds to PD-1. In certain embodiments, the soluble antibody, antigen-binding fragment, or fusion protein is an antagonist fusion protein that binds to PD-1. In certain embodiments, the soluble antibody, antigen-binding fragment, or fusion protein is an antagonist scFv that binds to PD-1. In certain embodiments, the soluble antibody, antigen-binding fragment, or fusion protein is an antagonist antibody, antigen-binding fragment, or fusion protein that binds to CTLA-4. In certain embodiments, the soluble antibody, antigen-binding fragment, or fusion protein is an antagonist scFv-Fc fusion protein that binds to CTLA-4. In certain embodiments, the soluble antibody, antigen-binding fragment, or fusion protein is an antagonist scFv that binds to CTLA-4. In certain embodiments, the soluble antibody, antigen-binding fragment, or fusion protein is an antagonist antibody, antigen-binding fragment, or fusion protein that binds to CD47.

In certain embodiments, the at least one soluble antibody, antigen-binding fragment, or fusion protein binds to a polypeptide having immunostimulatory activity. In certain embodiments, the polypeptide having immunostimulatory activity is selected from CD28, CD40, OX-40, 4-1BB, GITR, and their corresponding ligands or receptors (including, but not limited to, B7-1, B7-2, CD40L, OX-40L, 4-1BBL, and GITRL). In certain embodiments, the soluble antibody, antigen-binding fragment, or fusion protein is an agonist antibody, antigen-binding fragment, or fusion protein. In certain embodiments, the soluble antibody, antigen-binding fragment, or fusion protein is an agonist antibody, antigen-binding fragment, or fusion protein that binds to CD40. In certain embodiments, the soluble antibody, antigen-binding fragment, or fusion protein is an agonist scFv-Fc fusion protein that binds to CD40.

Cells comprising an antigen-recognizing receptor (e.g., a CAR) and a soluble scFv that binds a polypeptide that has immunosuppressive activity or immunostimulatory activity are disclosed in International Patent Publication No. WO 2014/134165, which is incorporated by reference herein in its entirety.

In certain embodiments, the cells comprising the presently disclosed GD3-targeted CAR further comprise two soluble antibodies, antigen-binding fragments, or fusion proteins. In certain embodiments, the two soluble antibodies, antigen-binding fragments, or fusion proteins are a soluble antibody, antigen-binding fragment, or fusion protein (e.g., an antagonist antibody, antigen-binding fragment, or fusion protein) that binds to a polypeptide having immunosuppressive activity and a soluble antibody, antigen-binding fragment, or fusion protein (e.g., an agonist antibody, antigen-binding fragment, or fusion protein) that binds to a polypeptide having immunostimulatory activity. In certain embodiments, the cells comprising the presently disclosed GD3-targeted CAR further comprise a soluble antibody, antigen-binding fragment, or fusion protein that binds to CD40 and a soluble antibody, antigen-binding fragment, or fusion protein that binds to PD-1. In certain embodiments, the cells comprising the presently disclosed GD3-targeted CAR further comprise a soluble agonist antibody, antigen-binding fragment, or fusion protein that binds to CD40 and a soluble antagonist antibody, antigen-binding fragment, or fusion protein that binds to PD-1. In certain embodiments, the cells comprising the presently disclosed GD3-targeted CAR further comprise a soluble agonist scFv-Fc fusion protein that binds to CD40 and a soluble antagonist scFv that binds to PD-1.

In certain embodiments, each of the two soluble antibodies, antigen-binding fragments, or fusion proteins binds to polypeptide having immunosuppressive activity. In certain embodiments, the cells comprising the presently disclosed GD3-targeted CAR further comprise a soluble antagonist antibody, antigen-binding fragment, or fusion protein that binds to PD-1 and a soluble antagonist antibody, antigen-binding fragment, or fusion protein that binds to CTLA-4. In certain embodiments, the cells comprising the presently disclosed GD3-targeted CAR further comprise a soluble antagonist scFv that binds to PD-1 and a soluble antagonist scFv-Fc fusion protein that binds to CTLA-4. In certain embodiments, the cells comprising the presently disclosed GD3-targeted CAR further comprise a soluble antagonist antibody, antigen-binding fragment, or fusion protein that binds to PD-1 and a soluble antagonist antibody, antigen-binding fragment, or fusion protein that binds to CD47.

CD40L

In certain embodiments, the cell further comprises an exogenous CD40L or a nucleic acid molecule encoding CD40L.

Cells comprising an antigen-recognizing receptor (e.g., a CAR) and an exogenous CD40L are disclosed in International Patent Publication No. WO 2014/134165, which is incorporated by reference herein in its entirety.

CD40 is expressed on antigen presenting cells such as dendritic cells. When stimulated by CD40 ligand (CD40L), dendritic cells become activated and provide a means to promote recruitment and activation of endogenous immune cells which may recognize additional antigens on tumor cells (“antigen spreading”). As disclosed in WO 2014/134165, CAR T cells that express CD40L are capable of activating dendritic cells and recruiting bystander T cells to promote antigen spreading. While systemic activation of CD40 is associated with toxicity, it has been shown that CAR T cells provide a means to deliver an antibody fragment locally in the tumor microenvironment while reducing systemic exposure (Rafiq et al. Nat Biotechnol (2018); 36:847-856). The inventors are in the development of GD3-targeted CAR T cells that are capable of activating CD40 either through expression of CD40L or an agonist antibody fragment.

Secreting Cytokines

In certain embodiments, the cell comprises at least one exogenous cytokine or a fragment thereof. In certain embodiments, the cell comprises a nucleic acid molecule encoding at least one exogenous cytokine or a fragment thereof. Non-limiting examples of cytokines include IL-12, IL-21, IL-2, IL-18, IL-33, and IL-36.

IL-18

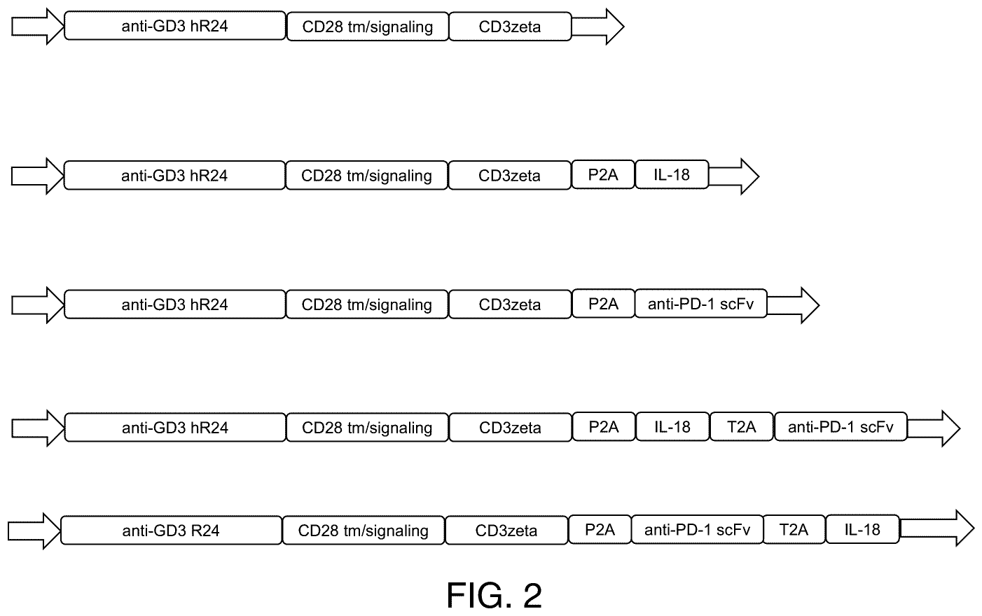

In certain embodiments, the cytokine is IL-18. In certain embodiments, the cell comprises an exogenous IL-18 polypeptide. See e.g., FIG. 2.

Interleukin 18 (also known as IGIF, IL-1g and IL1F4; GenBank ID: 3606 (human), 16173 (mouse), 29197 (rat), 403796 (dog), 100034216 (horse), 281249(cattle)) is a gene encoding a pro-inflammatory cytokine that increases immune activity of certain immunoresponsive cells. The protein product of Interleukin 18 includes, but is not limited to, NCBI Reference Sequences NP_001553.1 and NP_001230140.1. IL-18 is produced by macrophages, T cells and other cells. IL-18 functions by binding to the interleukin-18 receptor, and together with other cytokines, such as IL-12, it can induce cell-mediated immunity, which following infection with microbial products. After stimulation with IL-18, natural killer (NK) cells and certain T cells release other cytokines, such as interferon-γ (IFN-γ), IL-2 and TNF-α, which can further activate other types of immunoresponsive cells.

In certain embodiments, the term “IL-18” or “IL-18 cytokine” refers to the bioactive form of IL-18 after secretion from a cell (that is to say, where the signal peptide has been cleaved off).

In certain embodiments, the cell further comprises an exogenous IL-18 polypeptide. In certain embodiments, the exogenous IL-18 polypeptide comprises or consists of the amino acid sequence set forth in SEQ ID NO: 54, which is provided below. In certain embodiments, the exogenous IL-18 polypeptide comprises or consists of amino acids 23 to 179 of SE ID NO: 54.

[SEQ ID NO: 54]
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED

In certain embodiments, the cells further comprise a nucleic acid molecule encoding an IL-18 polypeptide. In certain embodiments, the nucleic acid molecule comprises the nucleotide sequence set forth in SEQ ID NO: 55, which is provided below.

[SEQ ID NO: 55]
TACTTCGGCAAGCTGGAGAGCAAGCTGAGCGTGATCAGGAACCTGAACGACCAGGTGCTGTTCATCGACCAGG
GCAACAGGCCCCTGTTCGAGGACATGACCGACAGCGACTGCAGGGACAACGCCCCCAGGACCATCTTCATCAT
CAGCATGTACAAGGACAGCCAGCCCAGGGGCATGGCCGTGACCATCAGCGTGAAGTGCGAGAAGATCAGCACC
CTGAGCTGCGAGAACAAGATCATCAGCTTCAAGGAGATGAACCCCCCCGACAACATCAAGGACACCAAGAGCG
ACATCATCTTCTTCCAGAGGAGCGTGCCCGGCCACGACAACAAGATGCAGTTCGAGAGCAGCAGCTACGAGGG
CTACTTCCTGGCCTGCGAGAAGGAGAGGGACCTGTTCAAGCTGATCCTGAAGAAGGAGGACGAGCTGGGCGAC
AGGAGCATCATGTTCACCGTGCAGAACGAGGAC

Alternatively, in certain embodiments, the cell further comprises a modified promoter/enhancer at an IL-18 gene locus, which can increase IL-18 gene expression, e.g., a constitutive or inducible promoter is placed to drive IL-18 gene expression.

Cells comprising an antigen-recognizing receptor (e.g., a CAR) and engineered to express IL-18, e.g., comprising an exogenous IL-18 polypeptide or a modified promoter/enhancer at an IL-18 gene locus are disclosed in International Patent Publication No. WO2018/027155, which is incorporated by reference herein in its entirety.

In certain embodiments, the cells comprising the presently disclosed GD3-targeted CAR further comprise a soluble scFv that binds to CD40 (e.g., an agonist scFv) and an exogenous IL-18 polypeptide.

In certain embodiments, the cells comprising the presently disclosed GD3-targeted CAR further comprise an exogenous IL-18 polypeptide and an exogenous CD40L.

In certain embodiments, the cells comprising the presently disclosed GD3-targeted CAR further comprise a soluble scFv that binds to PD-1 (e.g., an antagonist scFv) and an exogenous IL-18 polypeptide. See e.g., FIG. 2.

IL-36

In certain embodiments, the cytokine is IL-36. In certain embodiments, the cell comprises an exogenous IL-36 polypeptide.

IL-36 alpha, IL-36 beta and IL-36 gamma cytokines are produced by neutrophil, skin cells and other cells. They function by binding to the IL-36 receptor (IL-36R) and activate downstream signaling pathways. After stimulation with TL-36 alpha, TL-36 beta or TL-36 gamma, natural killer (NK) cells and certain T cells release other cytokines, such as IFN-γ, IL-10 and GM-CSF, which can further activate other types of immunoresponsive cells. After stimulation with TL-36 alpha, TL-36 beta or TL-36 gamma, dendritic cells can release IL-6, IL-12, CXCL1, CCL1, IL-23, and CXCL10, which can further modulate other types of immunoresponsive cells

Interleukin 36 alpha (IL-36 alpha) is also known as IL36A; FIL1; FILlE; IL1F6; IL-1F6; IL1(EPSILON); FIL1(EPSILON). GenBank ID: 27179 (human), 54448 (mouse), 296541 (rat), 523429 (cattle), 100065063 (horse). The protein product of IL-36 alpha includes, but is not limited to, NCBI Reference Sequences NP_055255.1, XP_011509267.1, XP_005263696.1 and XP_016859295.1.

Interleukin 36 beta (IL-36 beta) is also known as IL36B; FIL1; FIL1H; IL1F8; IL1H2; IL-1F8; IL-1H2; IL1-ETA; FIL1-(ETA); FILI-(ETA). GenBank ID: 27177 (human), 69677 (mouse), 362076 (rat), 100297786 (cattle), 483068 (dog), 100065096 (horse). The protein product of IL-36 beta includes, but is not limited to, NCBI Reference Sequences NP_055253.2, NP_775270.1 and XP_011509264.1.

Interleukin 36 gamma (IL-36 gamma) is also known as IL36G; IL1E; IL1F9; IL1H1; IL-1F9; IL-1H1; IL1RP2; IL-1RP2. GenBank ID: 56300 (human), 215257 (mouse), 499744 (rat), 615762 (cattle), 100686137 (dog), 100065031 (horse). The protein product of IL-36 gamma includes, but is not limited to, NCBI Reference Sequences NP_001265497.1 and NP_062564.1.

In certain embodiments, the term “IL-36” or “IL-36 cytokine” refers to the bioactive form of IL-36 alpha, IL-36 beta and/or IL-36 gamma after secretion from a cell (e.g., a form where the signal peptide is cleaved off).

In certain embodiments, the IL-36 polypeptide is a human IL-36 polypeptide.

In certain embodiments, the IL-36 polypeptide is a human IL-36 alpha polypeptide. In certain embodiments, the human IL-36 alpha polypeptide comprises or consists of the amino acid sequence set forth in SEQ ID NO: 56, which is provided below. In certain embodiments, the human IL-36 alpha polypeptide comprises or consists of an amino acid sequence that is at least about 80%, at least about 85%, at least about 90%, at least about 95%, at least about 99%, or at least about 100% homologous or identical to the sequence set forth in SEQ ID NO: 56.

[SEQ ID NO: 56]
KIDTPQQGSIQDINHRVWVLQDQTLIAVPRKDRMSPVTIALISCRHVET
LEKDRGNPIYLGLNGLNLCLMCAKVGDOPTLQLKEKDIMDLYNQPEPVK
SFLFYHSQSGRNSTFESVAFPGWFIAVSSEGGCPLILTQELGKANTTDF
GLTMLF

In certain embodiments, the IL-36 polypeptide is a human IL-36 beta polypeptide. In certain embodiments, the human IL-36 beta polypeptide comprises or consists of the amino acid sequence set forth in SEQ ID NO: 57, which is provided below. In certain embodiments, the human IL-36 beta polypeptide comprises or consists of an amino acid sequence that is at least about 80%, at least about 85%, at least about 90%, at least about 95%, at least about 99%, or at least about 100% homologous or identical to the sequence set forth in SEQ ID NO: 57.

[SEQ ID NO: 57]
REAAPKSYAIRDSRQMVWVLSGNSLIAAPLSRSIKPVTLHLIACRDTEF
SDKEKGNMVYLGIKGKDLCLFCAEIQGKPTLQLKLQGSQDNIGKDTCWK
LVGIHTCINLDVRESCFMGTLDQWGIGVGRKKWKSSFQHHHLRKKDKDF
SSMRTNIGMPGRM

In certain embodiments, the IL-36 polypeptide is a human IL-36 gamma polypeptide. In certain embodiments, the human IL-36 gamma polypeptide comprises or consists of the following amino acid sequence set forth in SEQ ID NO: 58, which is provided below. In certain embodiments, the human IL-36 gamma polypeptide comprises or consists of an amino acid sequence that is at least about 80%, at least about 85%, at least about 90%, at least about 95%, at least about 99%, or at least about 100% homologous or identical to the sequence set forth in SEQ ID NO: 58.

[SEQ ID NO: 58]
SMCKPITGTINDLNQQVWTLQGQNLVAVPRSDSVTPVTVAVITCKYPEA
LEQGRGDPIYLGIONPEMCLYCEKVGEQPTLQLKEQKIMDLYGQPEPVK
PFLFYRAKTGRTSTLESVAFPDWFIASSKRDQPIILTSELGKSYNTAFE
LNIND

In certain embodiments, the IL-36 polypeptide is a murine IL-36 polypeptide.

In certain embodiments, the IL-36 polypeptide is a murine IL-36 alpha polypeptide. In certain embodiments, the murine IL-36 alpha polypeptide comprises or consists of the amino acid sequence set forth in SEQ ID NO: 59, which is provided below. In certain embodiments, the murine IL-36 alpha polypeptide comprises or consists of an amino acid sequence that is at least about 80%, at least about 85%, at least about 90%, at least about 95%, at least about 99%, or at least about 100% homologous or identical to the sequence set forth in SEQ ID NO: 59.

[SEQ ID NO: 59]
GRETPDFGEVFDLDQQVWIFRNQALVTVPRSHRVTPVSVTILPCKYPES
LEQDKGIAIYLGIQNPDKCLFCKEVNGHPTLLLKEEKILDLYHHPEPMK
PFLFYHTRTGGTSTFESVAFPGHYIASSKTGNPIFLTSKKGEYYNINFN
LDIKS

In certain embodiments, the IL-36 polypeptide is a murine IL-36 beta polypeptide. In certain embodiments, the murine IL-36 beta polypeptide comprises or consists of the amino acid sequence set forth in SEQ ID NO: 60, which is provided below. In certain embodiments, the murine IL-36 beta polypeptide comprises or consists of an amino acid sequence that is at least about 80%, at least about 85%, at least about 90%, at least about 95%, at least about 99%, or at least about 100% homologous or identical to the sequence set forth in SEQ ID NO: 60.

[SEQ ID NO: 60]
SSQSPRNYRVHDSQQMVWVLTGNTLTAVPASNNVKPVILSLIACRDTEF
QDVKKGNLVFLGIKNRNLCFCCVEMEGKPTLQLKEVDIMNLYKERKAQK
AFLFYHGIEGSTSVFQSVLYPGWFIATSSIERQTIILTHQRGKLVNTNF
YIESEK

In certain embodiments, the IL-36 polypeptide is a murine IL-36 gamma polypeptide. In certain embodiments, the murine IL-36 gamma polypeptide comprises or consists of the amino acid sequence set forth in SEQ ID NO: 61, which is provided below. In certain embodiments, the murine IL-36 gamma polypeptide comprises or consists of an amino acid sequence that is at least about 80%, at least about 85%, at least about 90%, at least about 95%, at least about 99%, or at least about 100% homologous or identical to the sequence set forth in SEQ ID NO: 61.

[SEQ ID NO: 61]
GRETPDFGEVFDLDQQVWIFRNQALVTVPRSHRVTPVSVTILPCKYPES
LEQDKGIAIYLGIONPDKCLFCKEVNGHPTLLLKEEKILDLYHHPEPMK
PFLFYHTRTGGTSTFESVAFPGHYIASSKTGNPIFLTSKKGEYYNINEN
LDIKS

Alternatively, in certain embodiments, the cell further comprises a modified promoter/enhancer at an IL-36 gene locus, which can increase IL-36 gene expression, e.g., a constitutive or inducible promoter placed to drive IL-36 gene expression. Cells comprising an antigen-recognizing receptor (e.g., a CAR) and engineered to express IL-36, e.g., comprising an exogenous IL-36 polypeptide or a modified promoter/enhancer at an IL-36 gene locus are disclosed in International Patent Publication No. WO2019/099483, which is incorporated by reference herein in its entirety.

In certain embodiments, the cells comprising the presently disclosed GD3-targeted CAR further comprise a soluble antibody, a soluble antigen-binding fragment, or a fusion protein that binds to CD40 (e.g., an agonist antibody, antigen-binding fragment, or fusion protein, e.g., an agonist scFv-Fc fusion protein) and an exogenous IL-36 polypeptide.

In certain embodiments, the cells comprising the presently disclosed GD3-targeted CAR further comprise an exogenous IL-36 polypeptide (e.g., an exogenous IL-36 gamma polypeptide, e.g., an exogenous human IL-36 gamma polypeptide) and an exogenous CD40L.

In certain embodiments, the cells comprising the presently disclosed GD3-targeted CAR further comprise a soluble antibody, a soluble antigen-binding fragment, or a fusion protein that binds to PD-1 (e.g., an antagonist antibody, antigen-binding fragment, or fusion protein, e.g., an antagonist scFv) and an exogenous IL-36 polypeptide (e.g., an exogenous IL-36 gamma polypeptide, e.g., an exogenous human IL-36 gamma polypeptide).

In certain embodiments, the cells comprising the presently disclosed GD3-targeted CAR further comprise a soluble antibody, a soluble antigen-binding fragment thereof, or a fusion protein that binds to PD-1 (e.g., an antagonist antibody, antigen-binding fragment, or fusion protein, e.g., an antagonist scFv), an exogenous IL-36 polypeptide (e.g., an exogenous IL-36 gamma polypeptide, e.g., an exogenous human IL-36 gamma polypeptide), and an exogenous IL-18 polypeptide.

IL-33

In certain embodiments, the cytokine is IL-33. In certain embodiments, the cell comprises an exogenous IL-33 polypeptide.

Interleukin 33 (IL-33) (also known as DVS27; IL1F11; NF-HEV; NFEHEV; C9orf26; GenBank ID: 90865(human), 77125(mouse), 361749(rat), 507054(cattle), 100059908 (horse).) is a gene encoding a cytokine that binds to the IL1RL1/ST2 receptor. IL-33 is involved in the maturation of certain type of T cells and the activation of other immunoresponsive cells, such as mast cells, basophils, eosinophils and natural killer cells. The protein product of IL-33 includes, but is not limited to, NCBI Reference Sequences NP_001186569.1, NP_001186570.1, NP_001300973.1, NP_001300974.1, NP 001300975.1, NP_001300976.1, NP_001300977.1, NP_001340731.1, NP_254274.1, XP_016870774.1 and XP_011516363.1.

In certain embodiments, the term “IL-33” or “IL-33 cytokine” refers to the bioactive form of IL-33 after secretion from a cell (e.g., a form where the signal peptide is cleaved off).

In certain embodiments, the IL-33 polypeptide is a human IL-33 polypeptide. In certain embodiments, the human IL-33 polypeptide comprises or consists of the amino acid sequence set forth in SEQ ID NO: 62, which is provided below. In certain embodiments, the human IL-33 polypeptide comprises or consists of an amino acid sequence that is at least about 80%, at least about 85%, at least about 90%, at least about 95%, at least about 99%, or at least about 100% homologous or identical to the sequence set forth in SEQ ID NO: 62.

[SEQ ID NO: 62]
SITGISPITEYLASLSTYNDQSITFALEDESYEIYVEDLKKDEKKDKVL
LSYYESQHPSNESGDGVDGKMLMVTLSPTKDFWLHANNKEHSVELHKCE
KPLPDQAFFVLHNMHSNCVSFECKTDPGVFIGVKDNHLALIKVDSSENL
CTENILFKLSET

In certain embodiments, the IL-33 polypeptide is a murine IL-33 polypeptide. In certain embodiments, the murine IL-33 polypeptide comprises or consists of the amino acid sequence set forth in SEQ ID NO: 63, which is provided below. In certain embodiments, the murine IL-33 polypeptide comprises or consists of an amino acid sequence that is at least about 80%, at least about 85%, at least about 90%, at least about 95%, at least about 99%, or at least about 100% homologous or identical to the sequence set forth in SEQ ID NO: 63.

[SEQ ID NO: 63]
SIQGTSLLTQSPASLSTYNDQSVSFVLENGCYVINVDDSGKDQEQDQVL
LRYYESPCPASQSGDGVDGKKLMVNMSPIKDTDIWLHANDKDYSVELQR
GDVSPPEQAFFVLHKKSSDFVSFECKNLPGTYIGVKDNQLALVEEKDES
CNNIMFKLSKI

Alternatively, in certain embodiments, the cell further comprises a modified promoter/enhancer at an IL-33 gene locus, which can increase IL-33 gene expression, e.g., a constitutive or inducible promoter placed to drive IL-33 gene expression. Cells comprising an antigen-recognizing receptor (e.g., a CAR) and engineered to express IL-33, e.g., comprising an exogenous IL-33 polypeptide or a modified promoter/enhancer at an IL-33 gene locus are disclosed in International Patent Publication No. WO2019/099479, which is incorporated by reference in its entirety.

In certain embodiments, the cells comprising the presently disclosed GD3-targeted CAR further comprise a soluble antibody, a soluble antigen-binding fragment, or a fusion protein that binds to CD40 (e.g., an agonist antibody, antigen-binding fragment, or fusion protein, e.g., an agonist scFv-Fc fusion protein) and an exogenous IL-33 polypeptide.

In certain embodiments, the cells comprising the presently disclosed GD3-targeted CAR further comprise an exogenous IL-33 polypeptide and an exogenous CD40L.

In certain embodiments, the cells comprising the presently disclosed GD3-targeted CAR further comprise a soluble antibody, a soluble antigen-binding fragment, or a fusion protein that binds to PD-1 (e.g., an antagonist antibody, antigen-binding fragment, or fusion protein, e.g., an antagonist scFv) and an exogenous IL-33 polypeptide.

In certain embodiments, the cells comprising the presently disclosed GD3-targeted CAR further comprise a soluble antibody, a soluble antigen-binding fragment thereof, or a fusion protein that binds to PD-1 (e.g., an antagonist antibody, antigen-binding fragment thereof, or fusion protein, e.g., an antagonist scFv), an exogenous IL-33 polypeptide, and an exogenous IL-18 polypeptide.

### 5.5. Compositions and Vectors

The presently disclosed subject matter provides compositions comprising a presently disclosed GD3-targeted antigen-recognizing receptor (e.g., one disclosed in Section 5.3). Also provided are cells comprising such compositions.

In certain embodiments, the presently disclosed GD3-targeted antigen-recognizing receptor is operably linked to a promoter.

Furthermore, the present discloses subject matter provides nuclei acid compositions comprising a polynucleotide encoding a presently disclosed GD3-targeted antigen-recognizing receptor (e.g., one disclosed in Section 5.3). Also provided are cells comprising such nucleic acid compositions.

In certain embodiments, the nucleic acid composition further comprises a promoter that is operably linked to the presently disclosed GD3-targeted antigen-recognizing receptor.

In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and gamma retrovirus 5′ long-terminal repeat (LTR) promoter. In certain embodiments, the promoter is an inducible promoter. In certain embodiment, the inducible promoter is selected from the group consisting of a NFAT transcriptional response element (TRE) promoter, a CD69 promoter, a CD25 promoter, Nur77 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed GD3-targeted antigen-recognizing receptor (e.g., a CAR or a TCR like fusion molecule), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, the multicistronic expression cassette comprises a P2A peptide. In certain embodiments, the multicistronic expression cassette comprises a T2A peptide. In certain embodiments, the multicistronic expression cassette comprises a P2A peptide and a T2A peptide. Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.6. Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, CD8, CD28, CD3ζ, IL-18, IL-33, IL-36, etc.). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5.7. Formulations and Administration

The presently disclosed subject matter also provides compositions comprising the presently disclosed cells. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for inducing and/or enhancing an immune response to an antigen and/or treating and/or preventing a neoplasia. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. In certain embodiments, about 5×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, intravesicular administration, intracranial administration, intraocular administration, subcutaneous administration, intranodal administration, intratumoral administration, intrathecal administration, intrapleural administration, intraosseous administration, intraperitoneal administration, pleural administration, and direct administration to the subject.

### 5.8. Methods of Treatment

The presently disclosed subject cells and compositions comprising thereof can be used for treating and/or preventing a tumor or neoplasm. Such cells can be administered to a subject (e.g., a human subject) in need thereof for treatment and/or prevention of a tumor or neoplasm (e.g., sarcoma (e.g., soft tissue sarcoma (e.g., liposarcoma, and leiomyosarcoma) and bone sarcoma (e.g., osteosarcoma)), melanoma, lung cancer (e.g., small cell lung cancer (SCLC), non-small cell lung cancer (e.g., lung adenosarcoma), and Merkel cell carcinoma (MCC)). In certain embodiments, the cell is a T cell. The T cell can be a CD4⁺ T cell or a CD8⁺ T cell. In certain embodiments, the T cell is a CD4⁺ T cell.

The presently disclosed subject matter provides methods for inducing and/or increasing an immune response in a subject in need thereof. The presently disclosed cells and compositions comprising thereof can be used in a therapy or medicament. The presently disclosed subject matter provides various methods of using the cells (e.g., T cells) or compositions comprising thereof. For example, the presently disclosed cells and compositions comprising thereof can be used for reducing tumor burden in a subject. The presently disclosed cell can reduce the number of tumor cells, reduce tumor size, and/or eradicate the tumor in the subject. The presently disclosed cells and compositions comprising thereof can be used for treating and/or preventing a tumor or neoplasm in a subject. The presently disclosed cells and compositions comprising thereof can be used for prolonging the survival of a subject suffering from a tumor or neoplasm. Such methods comprise administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

The presently disclosed subject matter provides various methods of using the cells (e.g., T cells) or compositions comprising thereof. For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject.

The presently disclosed subject matter also provides methods of increasing or lengthening survival of a subject having a tumor or neoplasm. In certain embodiments, the method of increasing or lengthening survival of a subject having a tumor or neoplasm comprises administering the presently disclosed immunoresponsive cells or a composition comprising thereof to the subject. The method can reduce or eradicate tumor burden in the subject. Additionally, the presently disclosed subject matter provides methods for increasing an immune response in a subject, comprising administering the presently disclosed cell or a composition comprising thereof to the subject. The presently disclosed subject matter further provides methods for treating and/or preventing a tumor or neoplasm in a subject, comprising administering the presently disclosed cells or a composition comprising thereof to the subject.

In certain embodiments, the tumor or neoplasm is associated with ganglioside GD3. In certain embodiments, the tumor or neoplasm is associated with overexpression of ganglioside GD3. In certain embodiments, the tumor is cancer. In certain embodiments, the tumor or neoplasm is selected from the group consisting of sarcoma, melanoma, Merkel cell carcinoma (MCC), and lung cancer. In certain embodiments, the lung cancer is small cell lung cancer (SCLC) or non-small cell lung cancer. In certain embodiments, the non-small cell lung cancer comprises lung adenosarcoma.

In certain embodiments, the tumor or neoplasm is sarcoma. Non-limiting examples of sarcoma include bone sarcoma, soft tissue sarcoma, bone sarcoma (e.g., osteosarcoma), fibrosarcoma, myxosarcoma, chondrosarcoma, chordoma, angiosarcoma, endotheliosarcoma, lymphangiosarcoma, lymphangioendotheliosarcoma, synovioma, Ewing's tumor (or Ewing's sarcoma), leiomyosarcoma, rhabdomyosarcoma, and undifferentiated pleiomorphic sarcoma. In certain embodiments, the sarcoma is a soft tissue sarcoma. In certain embodiments, the soft tissue sarcoma is selected from the group consisting of liposarcoma, myxofibrosarcoma, and leiomyosarcoma. In certain embodiments, the sarcoma is bone sarcoma. In certain embodiments, the bone sarcoma comprises osteosarcoma.

In certain embodiments, the tumor or neoplasm is metastatic sarcoma. In certain embodiments, the tumor or neoplasm is melanoma. In certain embodiments, the tumor or neoplasm is malignant melanoma. In certain embodiments, the tumor or neoplasm is metastatic melanoma. In certain embodiments, the tumor or neoplasm is SCLC. In certain embodiments, the tumor or neoplasm is metastatic SCLC.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of a presently disclosed GD3-targeted antigen-recognizing receptor, adoptively transferred cells (e.g., immunoresponsive cells, e.g., T cells or NK cells) are endowed with augmented and selective cytolytic activity at the tumor site. Furthermore, subsequent to their localization to tumor or viral infection and their proliferation, the cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor (e.g., CAR). The suicide gene can be included within the vector comprising nucleic acids encoding a presently disclosed antigen-recognizing receptor (e.g., CAR). In this way, administration of a prodrug designed to activate the suicide gene (e.g., a prodrug (e.g., AP1903 that can activate iCasp-9) during malignant T-cell transformation or T cell-mediated toxicity (e.g., GVHD) triggers apoptosis in the suicide gene-activated cells expressing the antigen-recognizing receptor (e.g., CAR). The incorporation of a suicide gene into the a presently disclosed antigen-recognizing receptor (e.g., CAR) gives an added level of safety with the ability to eliminate the majority of receptor-expressing cells within a very short time period. A presently disclosed cell (e.g., a T cell) incorporated with a suicide gene can be pre-emptively eliminated at a given timepoint post the cell infusion, or eradicated at the earliest signs of toxicity.

### 5.9. Kits

The presently disclosed subject matter provides kits for inducing and/or enhancing an immune response in a subject, treating and/or preventing a tumor or neoplasm in a subject, reducing tumor burden in a subject, and/or increasing or lengthening survival of a subject having a tumor or neoplasm in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes a nucleic acid molecule encoding a presently disclosed GD3-targeted antigen-recognizing receptor (e.g., a CAR or a TCR like fusion molecule).

If desired, the cells and/or nucleic acid molecules are provided together with instructions for administering the cells or nucleic acid molecules to a subject having or at risk of developing a tumor or neoplasm. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Expression of Ganglioside GD3

The expression of GD3 in human tumor cell lines was measured. As shown in FIG. 1A, GD3 was expressed in melanoma, SCLC, osteosarcoma, liposarcoma, and leiomyosarcoma cell lines, but not in uterine sarcoma and ovarian cancer cell lines. GD3 expression was also detected in three lung adenocarcinoma cell lines and two Merkel cell carcinoma (MCC) cell lines.

### Example 2—GD3-Targeted CAR T Cells Kill Human SCLC, Melanoma, and Osteosarcoma Cells

T cell comprising a presently disclosed GD3-targeted CAR (hR24v) was developed. The proliferation and of hR24v-CAR T cells and were tested. Human T cells comprising hR24v-CAR T cells proliferated (see FIG. 1B) and secreted inflammatory cytokines (see FIG. 1C), when cultured with GD3⁺ tumor cells. hR24v-CAR T cells were effective at killing GD3⁺ melanoma, SCLC, and osteosarcoma cell lines in vitro. See FIGS. 1D and 1F. In addition, hR24v-CAR T cells shrank melanoma tumors in vivo in immunodeficient mice (see FIG. 1E). Furthermore, a full necropsy assessment with detailed histological analysis did not show any on-target, off-tumor toxicity and it was not evident that the hR24v-CAR T cells recognized GD3 on normal tissues. Thus, a GD3-targeted CAR provides a novel means to target GD3-positive tumors, including melanoma, bone sarcoma (e.g., osteosarcomas), soft tissue sarcoma (e.g., liposarcoma, and leiomyosarcoma), SCLC, etc.

### Example 3—Optimization of Dual-Armored GD3-Targeted CAR T Cells in Osteosarcoma, Melanoma, and SCLC

Validations of the hR2v4 CAR in osteosarcoma, melanoma, and SCLC are performed in vivo in xenografts of the osteosarcoma lines 143B and SaOS2, melanoma lines SK-MEL-28, and SCLC lines H69, H82, and DMS79.

R24 CAR constructs comprising “armors”, including IL-18 (Avanzi et al. Cell reports 23.7 (2018): 2130-2141), IL-36γ (Li et al., Leukemia (2020): 1-16), and a secreted anti-PD-1 antibody fragment (Rafiq et al. Nature biotechnology 36.9 (2018): 847-856), are prepared. CAR constructs comprising combinations of these armors are also prepared to protect CAR T cells from multiple mechanisms of suppression in the tumor microenvironment. For example, IL-18 secreted by CAR T cells is capable of repolarizing suppressive macrophages into an M1 phenotype (Avanzi et al. Cell reports 23.7 (2018): 2130-2141), while the anti-PD-1 antibody fragment can protect CAR T cells from suppressive PD-L1 expression on tumor cells (Rafiq et al. Nature biotechnology 36.9 (2018): 847-856), and CAR T cells with both armors (IL-18 and anti-PD-1 antibody) can be protected from both inhibitory pathways simultaneously. These constructs are also verified in vitro using the assays described in FIGS. 1A-D. Next, these constructs are tested in immunodeficient NSG mice engrafted with human osteosarcoma xenografts and anti-tumor efficacy is measured using bioluminescence imaging to identify the most optimal armored or dual-armored construct for subsequent characterization, with the ultimate aim of translating these constructs into Phase I clinical trials.

### Example 4—Characterization of Novel CD40-Targeted Armors in the Context of GD3-Targeted CAR T Cells in Osteosarcoma

CD40 is expressed on antigen-presenting cells such as dendritic cells. When stimulated by CD40 ligand (CD40L), dendritic cells become activated and provide a means to promote recruitment and activation of endogenous immune cells which may recognize additional antigens on tumor cells (“antigen spreading”). It was shown that CAR T cells that express CD40L are capable of activating dendritic cells and recruiting bystander T cells to promote antigen spreading (Kuhn et al., Cancer Cell 35.3 (2019): 473-488). CAR T cells targeting GD3 and secreting an activating CD40 antibody fragment are developed. While systemic activation of CD40 is associated with toxicity (Knorr et al., Proceedings of the National Academy of Sciences 115.43 (2018): 11048-11053), it was shown that CAR T cells provide a means to deliver an antibody fragment locally in the tumor microenvironment while reducing systemic exposure (Rafiq et al. Nature biotechnology 36.9 (2018): 847-856).

CD40 is expressed on some osteosarcomas and the consequences of activation of CD40 in osteosarcoma are unclear (Lollini et al., Clinical cancer research 4.8 (1998): 1843-1849; Zhang et al., Journal of bone oncology 17 (2019): 100245). hR24v-CAR T cells capable of activating CD40 either through the expression of CD40L (Kuhn et al., Cancer Cell 35.3 (2019): 473-488) or an agonist antibody fragment are generated. Importantly, human and mouse CD40/CD40L do not cross-react, and thus CD40 can be stimulated on either human osteosarcoma cells or endogenous mouse myeloid-derived cells separately. Thus, it is determined whether CD40 activation in either cell population has a pro-tumor or anti-tumor effect. Additional modifications of these CAR constructs comprising these agonistic agents (e.g., agonistic anti-CD40 antibody or expressing CD40L) are made to include other immune checkpoint armors including PD-1 and CTLA-4 to test whether these combinations increase efficacy. The CD40 agonists are combined with secreted IL-18 to assess the effects of this combination on both osteosarcoma and dendritic cell phenotypes.

### Example 5—GD3-Targeted CAR T Cells Comprising Secretable IL-18 Polypeptide and/or an Anti-PD-1 scFv

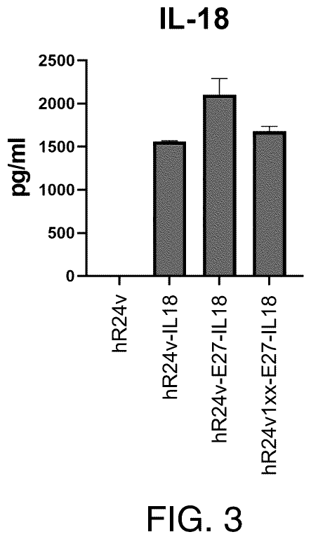

T cells comprising the presently disclosed R24 CAR with a secreteable IL-18 polypeptide and/or an anti-PD-1 scFv were also prepared (see FIG. 2). The secretion of IL-18 from these T cells was assessed. The results are shown in FIG. 3.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An antigen-recognizing receptor, comprising an extracellular antigen-binding domain, a transmembrane domain, and an intracellular signaling domain, wherein the extracellular antigen-binding domain specifically binds to ganglioside GD3 comprising a heavy chain variable region and a light chain variable region, wherein:
a) the heavy chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and
b) the light chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

2. The antigen-recognizing receptor of claim 1, wherein the extracellular antigen-binding domain is a single-chain variable fragment (scFv), a Fab, which is optionally crosslinked, or a F(ab)2.

3. The antigen-recognizing receptor of claim 2, wherein the extracellular antigen-binding domain is a humanized scFv.

4. The antigen-recognizing receptor of claim 2, wherein one or more of the scFv, Fab and F(ab)2 are comprised in a fusion protein with a heterologous sequence to form the extracellular antigen-binding domain.

5. The antigen-recognizing receptor of claim 1, wherein the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7; and/or the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8 or amino acids 1 to 107 of SEQ ID NO: 8.

6. The antigen-recognizing receptor of claim 1, wherein the heavy chain variable region comprises the amino acid sequence set forth in SEQ ID NO: 7, and the light chain variable region comprises the amino acid sequence set forth in SEQ ID NO: 8; or the heavy chain variable region comprises the amino acid sequence set forth in SEQ ID NO: 7, and the light chain variable region comprises amino acids 1 to 107 of SEQ ID NO: 8.

7. The antigen-recognizing receptor of claim 1, wherein a) the extracellular antigen-binding domain comprises a linker between the heavy chain variable region and the light chain variable region of the extracellular antigen-binding domain; b) a signal peptide is covalently joined to the 5′ terminus of the extracellular antigen-binding domain; and c) the heavy chain variable region and the light chain variable region are positioned from the N- to the C-terminus: V_{H}-V_{L}.

8. The antigen-recognizing receptor of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

9. The antigen-recognizing receptor of claim 1, wherein the intracellular signaling domain comprises a CD3ζ polypeptide.

10. The antigen-recognizing receptor of claim 1, wherein the intracellular signaling domain further comprises at least one co-stimulatory signaling region comprising a CD28 polypeptide, a 4-1BB polypeptide, an OX40 polypeptide, an ICOS polypeptide, a DAP-10 polypeptide, or a combination thereof.

11. The antigen-recognizing receptor of claim 1, wherein the antigen-recognizing receptor is a chimeric antigen receptor (CAR) or a T-cell receptor (TCR) like fusion protein.

12. An immunoresponsive cell comprising the antigen-recognizing receptor of claim 1.

13. The immunoresponsive cell of claim 12, wherein the antigen-recognizing receptor is constitutively expressed on the surface of the cell.

14. The immunoresponsive cell of claim 12, wherein a) the cell is a cell of the lymphoid lineage or a cell of the myeloid lineage; b) the cell is selected from the group consisting of a T cell, a Natural Killer (NK) cell, and a stem cell from which a lymphoid cell may be differentiated; c) the cell is a T cell; d) the cell is a cytotoxic T lymphocyte (CTL) or a regulatory T cell; e) the cell is a pluripotent stem cell; or f) the cell is an embryoid stem cell or an induced pluripotent stem cell.

15. The immunoresponsive cell of claim 12, wherein the cell is engineered to express at least one cytokine or a fragment thereof selected from the group consisting of IL-18, IL-36, IL-33, IL-12, IL-21, and IL-2.

16. The immunoresponsive cell of claim 15, wherein the cell comprises an exogenous IL-18 polypeptide, an exogenous IL-36 polypeptide, an IL-33 polypeptide, or a combination thereof.

17. The immunoresponsive cell of claim 12, wherein the cell comprises a soluble antibody, a soluble antigen-binding fragment, or a fusion protein, which binds to a polypeptide having immunosuppressive activity or immunostimulatory activity.

18. The immunoresponsive cell of claim 17, wherein a) the polypeptide having immunosuppressive activity is selected from the group consisting of CD47, PD-1, CTLA-4, BTLA, LAG-3, 2B4, CD47, SIRPα, PD-L1, PD-L2, TNFRSF14, CD48, and FGL-1; and b) the polypeptide having immunostimulatory activity is selected from the group consisting of CD28, CD40, OX-40, 4-1BB, B7-1, B7-2, CD40L, OX-40L, 4-1BBL, GITR, and GITRL.

19. The immunoresponsive cell of claim 18, wherein the soluble antibody, antigen-binding fragment, or fusion protein is a) an antagonist antibody, antigen-binding fragment, or fusion protein that binds to PD-1 or CTLA-4; b) an antagonist scFv that binds to PD-1 or an antagonist scFv-Fc fusion protein that binds to CTLA-4; c) an agonist antibody, antigen-binding fragment, or fusion protein that binds to CD40; and/or d) an agonist scFv-Fc fusion protein that binds to CD40.

20. The immunoresponsive cell of claim 12, wherein the cell comprises a) an exogenous IL-18 polypeptide and a soluble antibody, a soluble antigen-binding fragment, or a fusion protein that binds to PD-1; b) an exogenous IL-36 polypeptide and a soluble antibody, antigen-binding fragment, or fusion protein that binds to PD-1; c) an exogenous IL-18 polypeptide, an exogenous IL-36 polypeptide, and a soluble antibody, antigen-binding fragment, or fusion protein that binds to PD-1; or d) an exogenous IL-18 polypeptide and a soluble antibody, antigen-binding fragment, or fusion protein that binds to CD40.

21. The immunoresponsive cell of claim 12, wherein the cell further comprises a) an exogenous CD40L; b) an exogenous IL-18 polypeptide and an exogenous CD40L; and c) an exogenous IL-36 polypeptide and an exogenous CD40L.

22. A nucleic acid molecule encoding the antigen-recognizing receptor of claim 1.

23. A vector comprising the nucleic acid molecule of claim 22.

24. The vector of claim 23, wherein the vector is a retroviral vector, a γ-retroviral vector, or a lentiviral vector.

25. A host cell expressing the nucleic acid molecule of claim 22.

26. A composition comprising the cell of claim 12.

27. The composition of claim 26, which is a pharmaceutical composition further comprising a pharmaceutically acceptable carrier.

28. A method of reducing tumor burden in a subject having a tumor, increasing or lengthening survival of a subject having a tumor, and/or treating and/or preventing a tumor in a subject, the method comprising administering to the subject an effective amount of the cell of claim 12.

29. The method of claim 28, wherein the method reduces the number of tumor cells, reduces tumor size, and/or eradicates the tumor in the subject.

30. The method of claim 28, wherein the tumor is associated with ganglioside GD3.

31. The method of claim 28, wherein the tumor is selected from the group consisting of sarcoma, Merkel cell carcinoma (MCC), lung cancer, melanoma, bone sarcoma, soft tissue sarcoma, and small cell lung cancer (SCLC).

32. The method of claim 32, wherein a) the sarcoma is selected from the group consisting of bone sarcoma, soft tissue sarcoma, fibrosarcoma, myxosarcoma, chondrosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, and undifferentiated pleiomorphic sarcoma; and b) the bone sarcoma comprises osteosarcoma; and/or the soft tissue sarcoma is selected from the group consisting of liposarcoma, myxofibrosarcoma, and leiomyosarcoma.

33. A method for producing a cell comprising an antigen-recognizing receptor of claim 1, comprising introducing into the cell a nucleic acid molecule that encodes the antigen-recognizing receptor.

34. A kit for reducing tumor burden in a subject, treating and/or preventing a tumor in a subject, and/or increasing or lengthening survival of a subject having a tumor, comprising the cell of claim 12.
